(19) United States
(12) Patent Application Publication
(10) Pub. No.: US 2005/0267158 A1
(43) Pub. Date: Dec. 1, 2005

(54) Polymorphic and amorphous forms of 2,5-dimethyl-2H-pyrazole-3-carboxylic acid {2-fluoro-5-[3-((E)-2 pyridin-2-YL-vinyl)-1H-indazol-6-ylamino]-phenyl}-amide

(75) Inventors: Qiang Ye, San Diego, CA (US); Scott Edward Zook, San Diego, CA (US); Michael Ouellette, Descanso, CA (US); Don Hettinger, San Diego, CA (US); Jayaram K. Srirangam, San Diego, CA (US); Robert S. Kania, San Diego, CA (US); Nabil S. Saeed, San Diego, CA (US); Matthew D. Wightlin, San Diego, CA (US); Mark B. Mitchell, San Diego, CA (US)

Correspondence Address: Agouron Pharmaceuticals, Inc.;A Pfizer Company, Patent Department, 10777 Science Center Drive, San Diego, CA, 92121, US

(73) Assignee: AGOURON PHARMACEUTICALS, INC.

(21) Appl. No.: 11/081,708

(22) Filed: Mar. 15, 2005

Related U.S. Application Data
(60) Provisional application No. 60/554,140, filed on Mar. 17, 2004.

(51) Int. Cl. A61K 31/4439; C07D 43/14

(52) U.S. Cl. 514/338; 546/275.7

(57) ABSTRACT

The invention provides several polymorphic forms and an amorphous form of 2,5-Dimethyl-2H-pyrazole-3-carboxylic acid {2-fluoro-5-[3-((E)-2-pyridin-2-yl-vinyl)-1H-indazol-6-ylamino]-phenyl}-amide, pharmaceutical compositions containing such polymorphic or amorphous forms, and methods of using such pharmaceutical compositions to treat disease states mediated by protein kinases, such as cancer and other disease states associated with unwanted angiogenesis and/or cellular proliferation.

This application claims the benefit of U.S. Provisional Application Ser. No. 60/554,140, filed Mar. 17, 2004, the contents of which is hereby incoproated by reference in it's entirety.

### FIELD OF THE INVENTION

This invention relates to polymorphic and amorphous forms of 2,5-Dimethyl-2H-pyrazole-3-carboxylic acid {2-fluoro-5-[3-((E)-2-pyridin-2-yl-vinyl)-1H-indazol-6-ylamino]-phenyl}-amide, and to the therapeutic or prophylactic use of such compounds, and pharmaceutical compositions made therewith, to treat cancer and other disease states associated with unwanted angiogenesis and/or cellular proliferation.

### BACKGROUND OF THE INVENTION

U.S. Pat. No. 6,531,491, which is incorporated herein by reference in its entirety, is directed to indazole compounds that modulate and/or inhibit the activity of certain protein kinases. Such compounds are useful for treatment of cancer and other diseases associated with angiogenesis or cellular proliferation mediated by protein kinases. One compound disclosed in U.S. Pat. No. 6,531,491 is 2,5-Dimethyl-2H-pyrazole-3-carboxylic acid {2-fluoro-5-[3-((E)-2-pyridin-2-yl-vinyl)-1H-indazol-6-ylamino]-phenyl}-amide, the structure of which is shown below as Formula I:

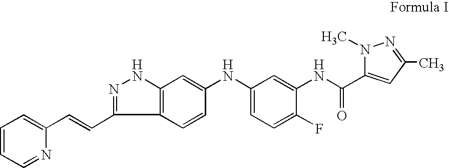

Another name for the compound of Formula I is 6-[N-(3-((1,3-Dimethyl-1H-pyrazol-5-yl)carboxamido)-4-fluoro-phenyl)amino]-3-E-[2-(pyridin-2-yl)ethenyl]-1H-indazole.

To prepare pharmaceutical compositions containing the compound of Formula I for administration to mammals in accordance with the requirements of U.S. and international health registration authorities (e.g., FDA's Good Manufacturing Practices (“GMP”)), there is a need to produce the compound of Formula I in a stable form, such as a stable crystalline form, having constant physical properties. Further, there is a need in the art to provide improved forms of the compound of Formula I having enhanced properties, such as improved solubility or oral bioavailability.

### SUMMARY OF THE INVENTION

In one aspect, the present invention provides fourteen polymorphic forms and one amorphous form of the compound of Formula I. In one embodiment, the invention provides a substantially pure polymorph of the compound shown in Formula I, where the polymorph is designated as Form I and has a powder X-ray diffraction (PXRD) pattern comprising the peaks at diffraction angles (2θ) of 5.5 and 28.4. More particularly, polymorph Form I has a PXRD pattern comprising the peaks at diffraction angles (2θ) of 5.5, 9.5, 10.7, and 28.4. Even more particularly, polymorph Form I has a PXRD pattern comprising the peaks at diffraction angles (2θ) essentially the same as shown in FIG. 1A. Still more particularly, polymorph Form I is characterized by a Raman spectra essentially the same as shown in FIG. 1C.

In another embodiment, the invention provides a substantially pure polymorph of the compound shown in Formula I, where the polymorph is designated as Form II and has a PXRD pattern comprising the peaks at diffraction angles (2%) of 12.1 and 16.7. More particularly, polymorph Form II has a PXRD pattern comprising the peaks at diffraction angles (2θ) of 12.1, 13.0, 16.7, and 18.3. Even more particularly, polymorph Form II has a PXRD pattern comprising the peaks at diffraction angles (2θ) essentially the same as shown in FIG. 2A. Still more particularly, polymorph Form II is characterized by a Raman spectra essentially the same as shown in FIG. 2C.

In another embodiment, the invention provides a substantially pure polymorph of the compound shown in Formula I, where the polymorph is designated as Form III and has a PXRD pattern comprising the peaks at diffraction angles (2θ) of 6.4 and 23.4. More particularly, polymorph Form III has a PXRD pattern comprising the peaks at diffraction angles (2θ) of 6.4, 23.4, 25.0, and 27.3. Even more particularly, polymorph Form III has a PXRD pattern comprising the peaks at diffraction angles (2θ) essentially the same as shown in FIG. 3A. Still more particularly, polymorph Form III is characterized by a Raman spectra essentially the same as shown in FIG. 3C.

In another embodiment, the invention provides a substantially pure polymorph of the compound shown in Formula I, where the polymorph is designated as Form IV and has a PXRD pattern comprising the peaks at diffraction angles (2θ) of 24.5 and 34.1. More particularly, polymorph Form IV has a PXRD pattern comprising the peaks at diffraction angles (2%) of 12.8, 15.8, 24.5, and 34.1. Even more particularly, polymorph Form IV has a PXRD pattern comprising the peaks at diffraction angles (2θ) essentially the same as shown in FIG. 4A. Still more particularly, polymorph Form IV is characterized by a Raman spectra essentially the same as shown in FIG. 4C. Even more particularly, polymorph Form IV can be characterized by an onset of crystal melting endotherm at about 118° C. at a scan rate of 10° C. per minute. Still more particularly, polymorph Form IV has a DSC thermogram essentially the same as shown in FIG. 4B.

In another embodiment, the invention provides a substantially pure polymorph of the compound shown in Formula I, where the polymorph is designated as Form V and has a PXRD pattern comprising the peaks at diffraction angles (2θ) of 8.4 and 26.0. More particularly, polymorph Form V has a PXRD pattern comprising the peaks at diffraction angles (2θ) of 8.4, 14.2, 22.2, and 26.0. Even more particularly, polymorph Form V has a PXRD pattern comprising the peaks at diffraction angles (2θ) essentially the same as shown in FIG. 5A. Still more particularly, polymorph Form IV is characterized by a Raman spectra essentially the same as shown in FIG. 5C.

In another embodiment, the invention provides a substantially pure polymorph of the compound shown in Formula I, where the polymorph is designated as Form Ia and has a PXRD pattern comprising the peaks at diffraction angles (2θ) of 5.5 and 25.2. More particularly, polymorph Form Ia has a PXRD pattern comprising the peaks at diffraction angles (2θ) of 5.5, 10.6, 18.9, and 25.2. Even more particularly, polymorph Form Ia has a PXRD pattern comprising the peaks at diffraction angles (2θ) essentially the same as shown in FIG. 6A.

In another embodiment, the invention provides a substantially pure polymorph of the compound shown in Formula I, where the polymorph is designated as Form Ib and has a PXRD pattern comprising the peaks at diffraction angles (2θ) of 10.2 and 13.8. More particularly, polymorph Form Ib has a PXRD pattern comprising the peaks at diffraction angles (2θ) of 10.2, 13.8, 20.1, and 26.2. Even more particularly, polymorph Form Ib has a PXRD pattern comprising the peaks at diffraction angles (2θ) essentially the same as shown in FIG. 7A. Still more particularly, polymorph Form Ib is characterized by a Raman spectra essentially the same as shown in FIG. 7C.

In another embodiment, the invention provides a substantially pure polymorph of the compound shown in Formula I, where the polymorph is designated as Form IIa and has a PXRD pattern comprising the peaks at diffraction angles (2θ) of 12.8 and 22.9. More particularly, polymorph Form IIa has a PXRD pattern comprising the peaks at diffraction angles (2θ) of 12.8, 16.0, 22.9, and 31.2. Even more particularly, polymorph Form IIa has a PXRD pattern comprising the peaks at diffraction angles (2θ) essentially the same as shown in FIG. 8A.

In another embodiment, the invention provides a substantially pure polymorph of the compound shown in Formula I, where the polymorph is designated as Form IIb and has a PXRD pattern comprising the peaks at diffraction angles (2θ) of 14.3 and 19.0. More particularly, polymorph Form IIb has a PXRD pattern comprising the peaks at diffraction angles (2θ) of 7.9, 14.3, 19.0, and 27.0. Even more particularly, polymorph Form IIb has a PXRD pattern comprising the peaks at diffraction angles (2θ) essentially the same as shown in FIG. 9A. Still more particularly, polymorph Form IV is characterized by a Raman spectra essentially the same as shown in FIG. 9C.

In another embodiment, the invention provides a substantially pure polymorph of the compound shown in Formula I, where the polymorph is designated as Form IIIa and has a PXRD pattern comprising the peaks at diffraction angles (2θ) of 24.9 and 36.2. More particularly, polymorph Form IIIa has a PXRD pattern comprising the peaks at diffraction angles (2θ) of 14.7, 21.0, 24.9, and 36.2. Even more particularly, polymorph Form IIIa has a PXRD pattern comprising the peaks at diffraction angles (2θ) essentially the same as shown in FIG. 10A.

In another embodiment, the invention provides a substantially pure polymorph of the compound shown in Formula I, where the polymorph is designated as Form IIIb and has a PXRD pattern comprising the peaks at diffraction angles (28) of 6.8 and 14.5. More particularly, polymorph Form IIIb has a PXRD pattern comprising the peaks at diffraction angles (2%) of 6.8, 14.5, 20.8, and 24.8. Even more particularly, polymorph Form IIIb has a PXRD pattern comprising the peaks at diffraction angles (2θ) essentially the same as shown in FIG. 11A. Still more particularly, polymorph Form IIIb is characterized by a Raman spectra essentially the same as shown in FIG. 11C.

In another embodiment, the invention provides a substantially pure polymorph of the compound shown in Formula I, where the polymorph is designated as Form IVa and has a PXRD pattern comprising the peaks at diffraction angles (2θ) of 13.5 and 32.5. More particularly, polymorph Form IVa has a PXRD pattern comprising the peaks at diffraction angles (2θ) of 13.5, 15.8, 27.0, and 32.5. Even more particularly, polymorph Form IVa has a PXRD pattern comprising the peaks at diffraction angles (2θ) essentially the same as shown in FIG. 12A. Still more particularly, polymorph Form IVa has an onset of dehydration endotherm at about 63° C. and an onset of crystal melting endotherm at about 123° C. at a scan rate of 10° C. per minute. Still further, polymorph Form IVa has a DSC thermogram essentially the same as shown in FIG. 12B.

In another embodiment, the invention provides a substantially pure polymorph of the compound shown in Formula I, where the polymorph is designated as Form Va and has a PXRD pattern comprising the peaks at diffraction angles (2θ) of 19.2 and 33.9. More particularly, polymorph Form Va has a PXRD pattern comprising the peaks at diffraction angles (2θ) of 11.5, 19.2, 24.4, and 33.9. Even more particularly, polymorph Form Va has a PXRD pattern comprising the peaks at diffraction angles (2θ) essentially the same as shown in FIG. 13A. Still more particularly, polymorph Form Va is characterized by a Raman spectra essentially the same as shown in FIG. 13C.

In another embodiment, the invention provides a substantially pure polymorph of the compound shown in Formula I, where the polymorph is designated as Form VI and has a PXRD pattern comprising the peaks at diffraction angles (2θ) of 7.7 and 26.8. More particularly, polymorph Form VI has a PXRD pattern comprising the peaks at diffraction angles (2θ) of 7.7, 12.9, 18.5, and 26.8. Even more particularly, polymorph Form VI has a PXRD pattern comprising the peaks at diffraction angles (2θ) essentially the same as shown in FIG. 14A. Still more particularly, polymorph Form VI is characterized by a Raman spectra essentially the same as shown in FIG. 14C.

In another embodiment, the invention provides an amorphous form of the compound shown in Formula I, where the amorphous form has a PXRD pattern exhibiting a broad peak at diffraction angles (2θ) ranging from 4 to 400 without any of the sharp peaks characteristic of a crystalline form. More particularly, the amorphous form is characterized by having a PXRD pattern essentially the same as shown in FIG. 15A. Even more particularly, the amorphous form is characterized by a Raman spectra comprising shift peaks (cm⁻¹) essentially the same as shown in FIG. 15B.

In yet another embodiment, the invention provides a solid form of the compound shown in Formula I, wherein the solid form is a mixture comprising at leat two of the following solid forms: polymorph Forms I, II, III, IV, V, Ia, Ib, IIa, IIb, IIIa, IIIb, IVa, Va, VI, and an amorphous form.

In another embodiment, the invention provides a substantially pure polymorph of the compound shown in Formula I, where the polymorph is designated as Form Ibm-2, which is a mixture of Forms Ib and VI, and has a PXRD pattern comprising the peaks at diffraction angles (2θ) of 12.9 and 13.8. More particularly, polymorph Form Ibm-2 has a PXRD pattern comprising the peaks at diffraction angles (2θ) of 12.9, 13.8, 20.1, and 26.8. Even more particularly, polymorph Form Ibm-2 has a PXRD pattern comprising the peaks at diffraction angles (2θ) essentially the same as shown in FIG. 16.

In another aspect, the invention relates to pharmaceutical compositions, each comprising a crystalline or amorphous form of the compound of Formula I. The invention also relates to a pharmaceutical composition comprising a mixture of at least two of any of the polymorphic and amorphous forms.

In a further aspect, the invention provides methods of treating cancer as well as other disease states associated with unwanted angiogenesis and/or cellular proliferation, comprising administering a therapeutically effective amount of a polymorph/amorphous compound of the invention to a patient in need of such treatment. The invention also relates to a method of modulating and/or inhibiting the kinase activity of VEGF-R (vascular endothelial cell growth factor receptor), FGF-R (fibroblast growth factor receptor), a CDK (cyclin-dependent kinase) complex, CHK1, LCK (also known as lymphocyte-specific tyrosine kinase), TEK (also known as Tie-2), FAK (focal adhesion kinase), and/or phosphorylase kinase by administering a compound of the invention. Preferred compounds of the present invention have selective kinase activity—i.e., they possess significant activity against one or more specific kinases while possessing less or minimal activity against one or more different kinases. In one preferred embodiment of the invention, polymorph/amorphous compounds of the present invention are those possessing substantially higher potency against VEGF receptor tyrosine kinase than against FGF-R1 receptor tyrosine kinase. The invention is also directed to methods of modulating VEGF receptor tyrosine kinase activity without significantly modulating FGF receptor tyrosine kinase activity.

The compounds of the invention may be used advantageously in combination with other known therapeutic agents. For example, the polymorph/amorphous forms of the compound of Formula I that possess anti-angiogenic activity may be co-administered with cytotoxic chemotherapeutic agents, such as taxol, taxotere, vinblastine, cis-platin, doxorubicin, adriamycin, and the like, to produce an enhanced antitumor effect. Additive or synergistic enhancement of therapeutic effect may also be obtained by co-administration of a compound of the invention that possesses anti-angiogenic activity with other anti-angiogenic agents, such as combretastatin A-4, endostatin, prinomastat, celecoxib, rofocoxib, EMD121974, IM862, anti-VEGF monoclonal antibodies, and anti-KDR monoclonal antibodies.

### BRIEF DESCRIPTION OF THE DRAWINGS

Having thus described the invention in general terms, reference will now be made to the accompanying drawings, wherein:

FIG. 1A is an X-ray powder diffraction diagram of polymorph Form I of the invention;

FIG. 1B is a Differential Scanning Calorimetry (DSC) thermogram of polymorph Form I of the invention;

FIG. 1C is a Raman spectral diagram of polymorph Form I of the invention;

FIG. 2A is an X-ray powder diffraction diagram of polymorph Form II of the invention;

FIG. 2B is a DSC thermogram of polymorph Form II of the invention;

FIG. 2C is a Raman spectral diagram of polymorph Form II of the invention;

FIG. 3A is an X-ray powder diffraction diagram of polymorph Form III of the invention;

FIG. 3B is a DSC thermogram of polymorph Form III of the invention;

FIG. 3C is a Raman spectral diagram of polymorph Form III of the invention;

FIG. 4A is an X-ray powder diffraction diagram of polymorph Form IV of the invention;

FIG. 4B is a DSC thermogram of polymorph Form IV of the invention;

FIG. 4C is a Raman spectral diagram of polymorph Form IV of the invention;

FIG. 5A is an X-ray powder diffraction diagram of polymorph Form V of the invention;

FIG. 5B is a DSC thermogram of polymorph Form V of the invention;

FIG. 5C is a Raman spectral diagram of polymorph Form V of the invention;

FIG. 6A is an X-ray powder diffraction diagram of polymorph Form Ia of the invention;

FIG. 6B is a DSC thermogram of polymorph Form Ia of the invention;

FIG. 7A is an X-ray powder diffraction diagram of polymorph Form Ib of the invention;

FIG. 7B is a DSC thermogram of polymorph Form Ib of the invention;

FIG. 7C is a Raman spectral diagram of polymorph Form Ib of the invention;

FIG. 8A is an X-ray powder diffraction diagram of polymorph Form IIa of the invention;

FIG. 8B is a DSC thermogram of polymorph Form IIa of the invention;

FIG. 9A is an X-ray powder diffraction diagram of polymorph Form IIb of the invention;

FIG. 9B is a DSC thermogram of polymorph Form IIb of the invention;

FIG. 9C is a Raman spectral diagram of polymorph Form IIb of the invention;

FIG. 10A is an X-ray powder diffraction diagram of polymorph Form IIIa of the invention;

FIG. 11A is an X-ray powder diffraction diagram of polymorph Form IIIb of the invention;

FIG. 11B is a DSC thermogram of polymorph Form IIIb of the invention;

FIG. 11C is a Raman spectral diagram of polymorph Form IIIb of the invention;

FIG. 12A is an X-ray powder diffraction diagram of polymorph Form IVa of the invention;

FIG. 12B is a DSC thermogram of polymorph Form IVa of the invention;

FIG. 13A is an X-ray powder diffraction diagram of polymorph Form Va of the invention;

FIG. 13B is a DSC thermogram of polymorph Form Va of the invention;

FIG. 13C is a Raman spectral diagram of polymorph Form Va of the invention;

FIG. 14A is an X-ray powder diffraction diagram of polymorph Form VI of the invention;

FIG. 14B is a DSC thermogram of polymorph Form VI of the invention;

FIG. 14C is a Raman spectral diagram of polymorph Form VI of the invention;

FIG. 15A is an X-ray powder diffraction diagram of an amorphous form of the invention;

FIG. 15B is a Raman spectral diagram of an amorphous form of the invention; and

FIG. 16 is an X-ray powder diffraction diagram of polymorph Form Ibm-2 of the invention.

### DETAILED DESCRIPTION OF THE INVENTION

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art.

I. Definitions

The following terms as used herein have the meanings indicated.

As used in the specification, and in the appended claims, the singular forms “a”, “an”, “the”, include plural referents unless the context clearly dictates otherwise.

The terms “comprising” and “including” are used in an open, non-limiting sense.

The term “polymorph” refers to a crystalline form of a compound with a distinct spatial lattice arrangement as compared to other crystalline forms of the same compound.

The term “amorphous” refers to a non-crystalline form of a compound.

“A pharmaceutically acceptable salt” is intended to mean a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, y-hydroxybutyrates, glycollates, tartrates, methane-sulfonates, propanesulfonates, naphthalene-1-sulfonates, naphthalene-2-sulfonates, and mandelates.

II. Polymorphic and Amorphous Forms of the Compound of Formula I

The present invention provides several polymorph crystalline forms and an amorphous form of the compound of Formula I. Each crystalline or amorphous form of the compound can be characterized by one or more of the following: X-ray powder diffraction pattern (i.e., X-ray diffraction peaks at various diffraction angles (2θ)), melting point onset (and onset of dehydration for hydrated forms) as illustrated by endotherms of a Differential Scanning Calorimetry (DSC) thermogram, Raman spectral diagram pattern, aqueous solubility, light stability under International Conference on Harmonization (ICH) high intensity light conditions, and physical and chemical storage stability.

The X-ray powder diffraction pattern for each polymorph or amorphous form of the invention was measured on a Shimadzu XRD-6000 X-ray diffractometer equipped with a Cu X-ray source operated at 40 kV and 50 mA. Samples were placed in a sample holder and then packed and smoothed with a glass slide. During analysis, the samples were rotated at 60 rpm and analyzed from angles of 4 to 40° (θ-2θ) at 5°/min with a 0.04° step or at 2°/min with a 0.02° step. If limited material was available, samples were placed on a silicon plate (zero background) and analyzed without rotation. One of skill in the art will appreciate that the peak positions (2θ) will show some inter-apparatus variability, typically as much as 0.1°. Accordingly, where the solid forms of the present invention are described as having a powder X-ray diffraction pattern essentially the same as that shown in a given figure, the term “essentially the same” is intended to encompass such inter-apparatus variability in diffraction peak positions.

The DSC thermographs were obtained using a Mettler Toledo DSC821^{e} instrument at a scan rate of 10° C./min over a temperature range of 30-250° C. Samples were weighed into 40 μl aluminum crucibles that were sealed and punctured with a single hole. The extrapolated onset of melting temperature and, where applicable, the onset of dehydration temperature, were calculated.

Depending on several factors, the endotherms exhibited by the compounds of the invention may vary (by about 0.01-5° C. for crystal polymorph melting and by about 0.01-20° C. for polymorph dehydration) above or below the endotherms depicted in the appended figures. Factors responsible for such variance include the rate of heating (i.e., the scan rate) at which the DSC analysis is conducted, the way the DSC onset temperature is defined and determined, the calibration standard used, instrument calibration, the relative humidity and the chemical purity of the sample. For any given sample, the observed endotherms may also differ from instrument to instrument; however, it will generally be within the ranges defined herein provided the instruments are calibrated similarly.

Raman scattering spectra were obtained by using a Fourier transform Raman spectrophotometer Kaiser Optical Instruments, Ramen RXN1-785. The excitation light source was a Invictus NIR Laser operating at 785 nm wavelength. The detector was an Andor CCD. The resolution was 34 cm⁻¹.

The polymorph or amorphous forms of the invention are preferably substantially pure, meaning each polymorph or amorphous form of the compound of Formula I includes less than 10%, preferably less than 5%, preferably less than 3%, preferably less than 1% by weight of impurities, including other polymorph or amorphous forms of the compound.

The solid forms of the present invention may also exist together in a mixture. Mixtures of polymorphs and/or the amorphous form of the present invention will have X-ray diffraction peaks characteristic of each of the polymorphs and/or amorphous forms present in the mixture. For example, a mixture of two polymorphs will have a powder X-ray diffraction pattern that is a convolution of the X-ray diffraction patterns corresponding to the substantially pure polymorphs.

A. PolymorDh Form I

Polymorph Form I, an anhydrous form, can be prepared by slurrying the compound of Formula I in ethanol and then heating to reflux for 30 minutes, followed by slow cooling to 23° C. Form I has an aqueous solubility of about 39 μg/mL at pH 2 and about 0.4 μg/mL at pH 7.4. Form I is light-stable under ICH high intensity light conditions and chemically stable at 80° C. and 40° C./75% RH for at least 14 days.

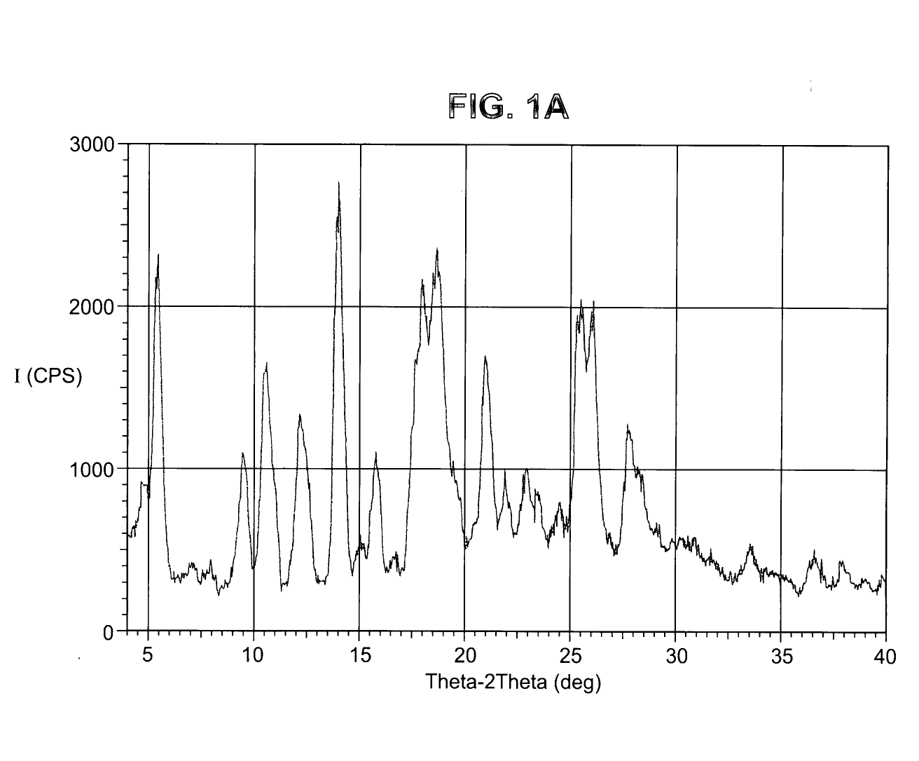

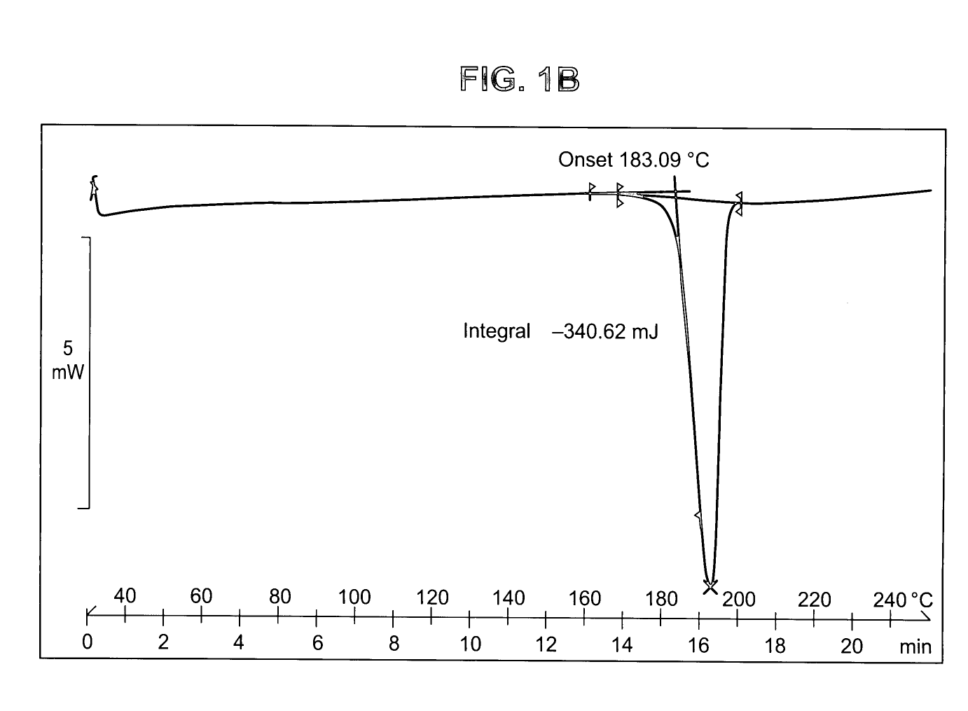

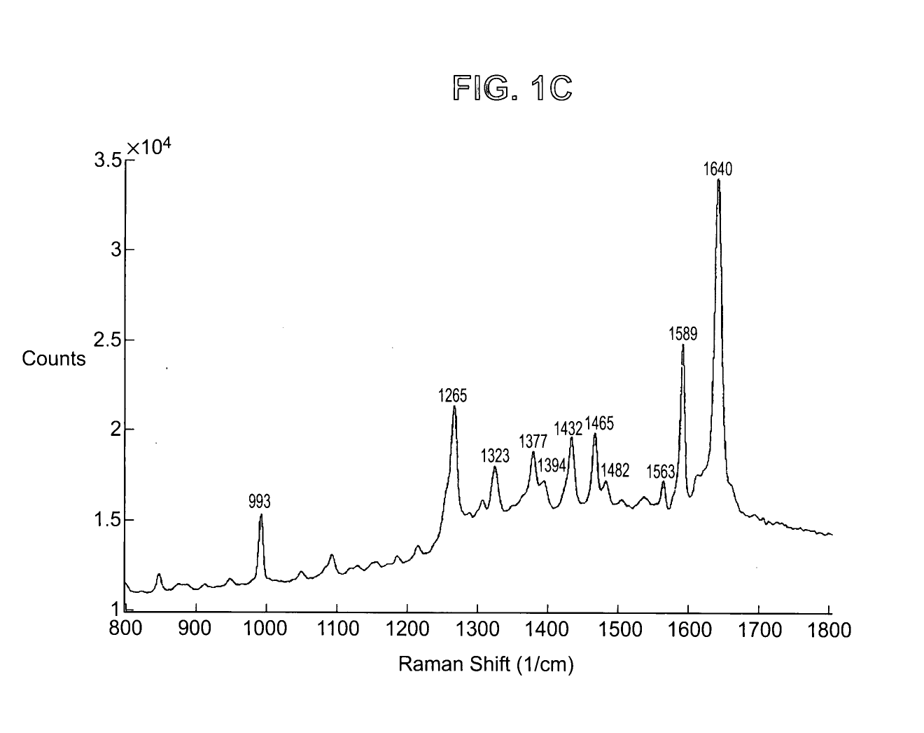

Form I is characterized by an X-ray powder diffraction pattern with peaks at the following approximate diffraction angles (2θ): 4.80, 5.49, 7.06, 7.90, 9.52, 10.67, 12.33, 14.10, 15.08, 15.80, 18.12, 18.80, 19.72, 20.40, 21.09, 21.95, 23.00, 23.48, 24.52, 25.52, 26.16, 27.92, 28.36, 29.08, 29.88, 30.32, 30.96, 31.68, 33.59, 34.32, 34.72, 35.20, 36.64, and 38.00. FIG. 1A provides an X-ray powder diffraction pattern for Form I.

The DSC thermogram for Form I, shown in FIG. 1B, indicates an onset of crystal melting endotherm at about 183° C., at a scan rate of 10° C./minute.

The Raman spectral diagram for Form I, shown in FIG. 1C, includes Raman Shift peaks (cm⁻¹) at approximately 993, 1265, 1323, 1377, 1394, 1432, 1465, 1482, 1563, 1589, and 1640.

B. Polymorph Form II

Polymorph Form II, an anhydrous form, can be prepared by direct crystallization of a solution of the compound of Formula I in tetrahydrofuran at 60° C. by hexanes. Form II has an aqueous solubility of about 19 μg/mL at pH 2 and about 0.7 μg/mL at pH 7.4. Form II is light-stable under ICH high intensity light conditions.

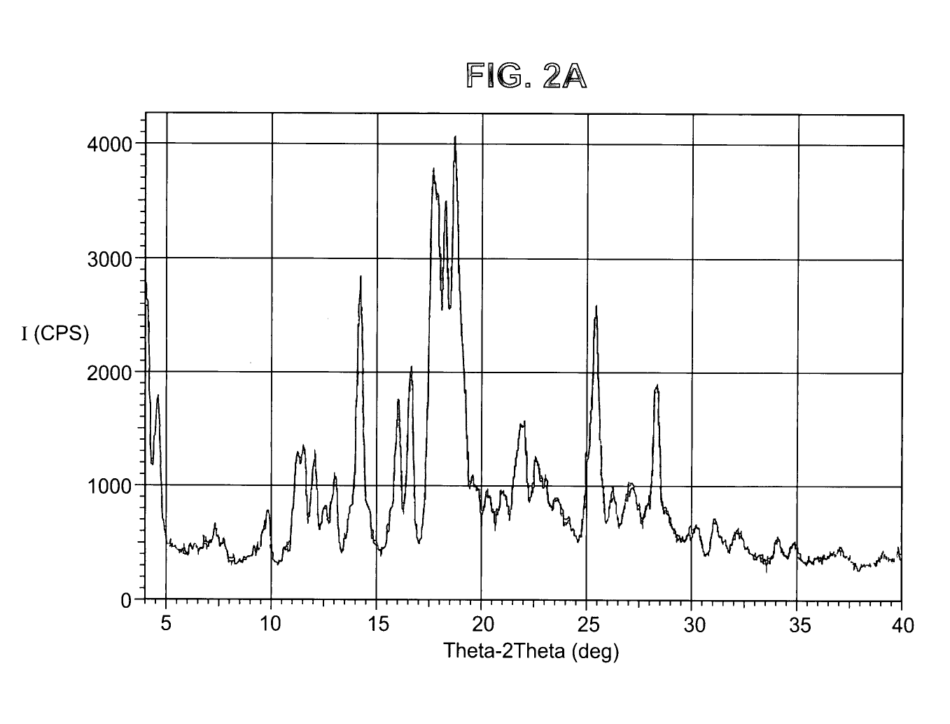

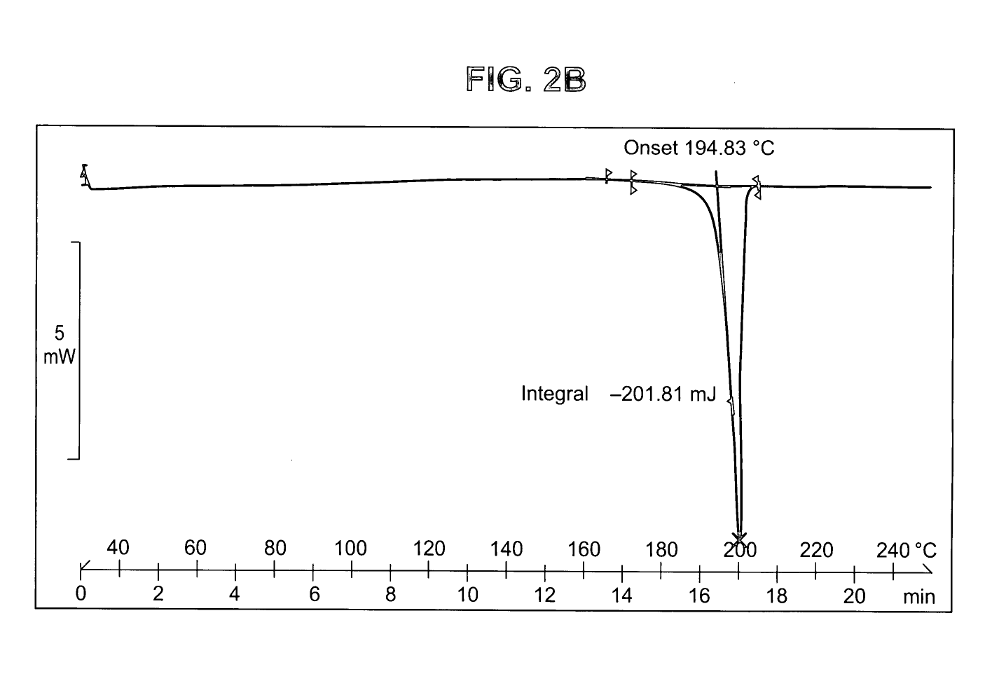

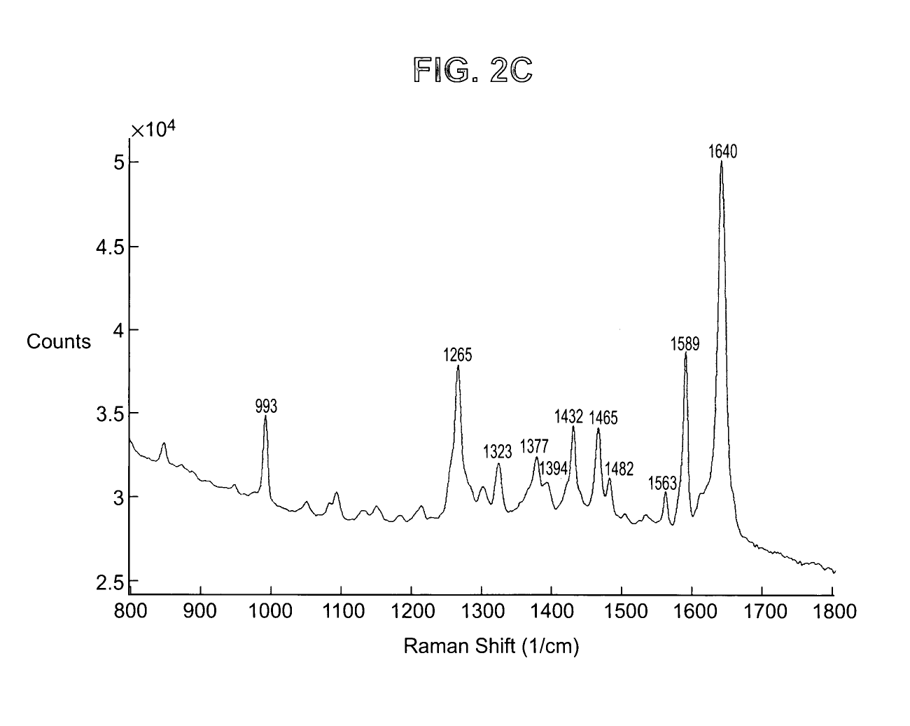

Form II is characterized by an X-ray powder diffraction pattern with peaks at the following approximate diffraction angles (2θ): 4.65, 6.9200, 7.36, 7.76, 9.81, 11.41, 12.08, 12.60, 13.03, 13.72, 14.24, 14.72, 16.06, 16.66, 17.80, 18.32, 18.80, 19.68, 20.32, 21.05, 21.89, 22.64, 23.00, 23.60, 25.45, 26.30, 27.18, 28.34, 29.04, 30.21, 31.14, 32.24, 34.14, 34.91, 36.97, 39.21, and 39.92. FIG. 2A provides an X-ray powder diffraction pattern for Form II.

The DSC thermogram for Form II, shown in FIG. 2B, indicates an onset of crystal melting endotherm at about 195° C., at a scan rate of 10° C./minute.

The Raman spectral diagram for Form II, shown in FIG. 2C, includes Raman Shift peaks (cm⁻¹) at approximately 993, 1265, 1323, 1377, 1394, 1432, 1465, 1482, 1563, 1589, and 1640.

C. Polymorph Form III

Polymorph Form III, an anhydrous form, can be prepared by slurrying Form I solids in mineral oil at 192° C. for about 1.5 hours, followed by hexane wash and filtration. Form III has an aqueous solubility of about 10 μg/mL at pH 2 and about 0.6 μg/mL at pH 7.4. Form III is light-stable under ICH high intensity light conditions.

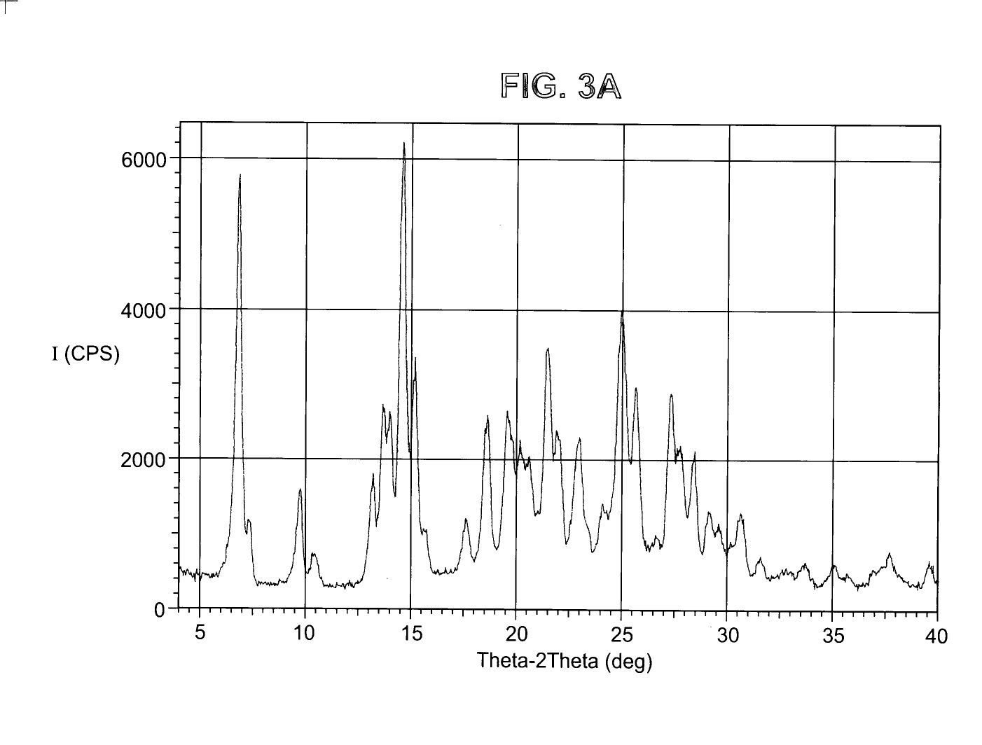

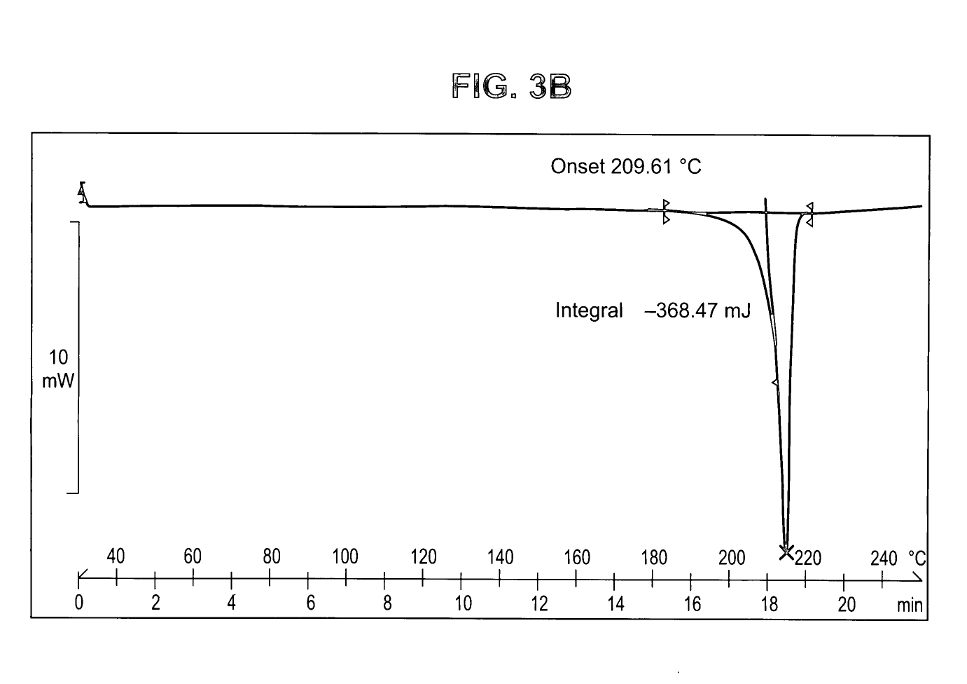

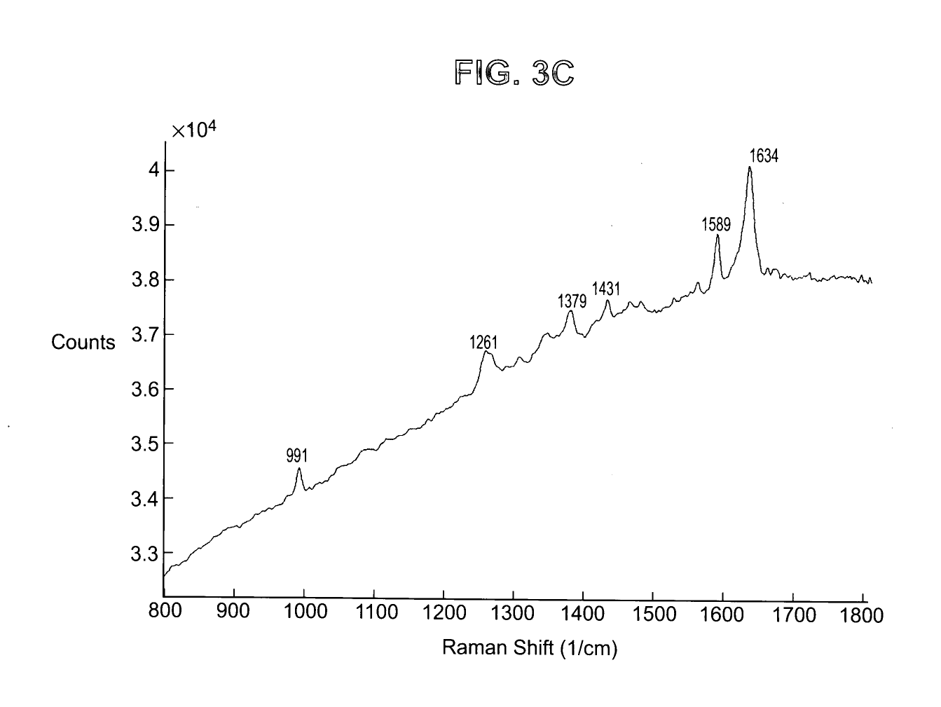

Form III is characterized by an X-ray powder diffraction patterri with peaks at the following approximate diffraction angles (2θ): 6.40, 6.87, 7.36, 9.73, 10.43, 13.20, 13.72, 14.04, 14.65, 15.20, 15.80, 17.60, 18.56, 19.56, 20.16, 20.56, 21.49, 21.96, 22.92, 23.40, 24.08, 24.98, 25.64, 27.32, 27.72, 28.35, 29.08, 29.56, 30.12, 30.58, 31.53, 33.58, 35.01, 36.84, 37.24, 37.60, and 39.51. FIG. 3A provides an X-ray powder diffraction pattern for Form III.

The DSC thermogram for Form III, shown in FIG. 3B, indicates an onset of crystal melting endotherm at about 210° C., at a scan rate of 10° C./minute.

The Raman spectral diagram for Form III, shown in FIG. 3C, includes Raman Shift peaks (cm⁻¹) at approximately 991, 1261, 1379, 1431, 1589, and 1634.

D. Polymorph Form IV

Form IV, an anhydrous form, can be prepared by crystallization of the compound of Formula I in ethyl acetate and ethanol by 1:1 NaHCO₃:water. Form IV has an aqueous solubility of about 7 μg/mL at pH 2. Form IV is light-stable under ICH high intensity light conditions.

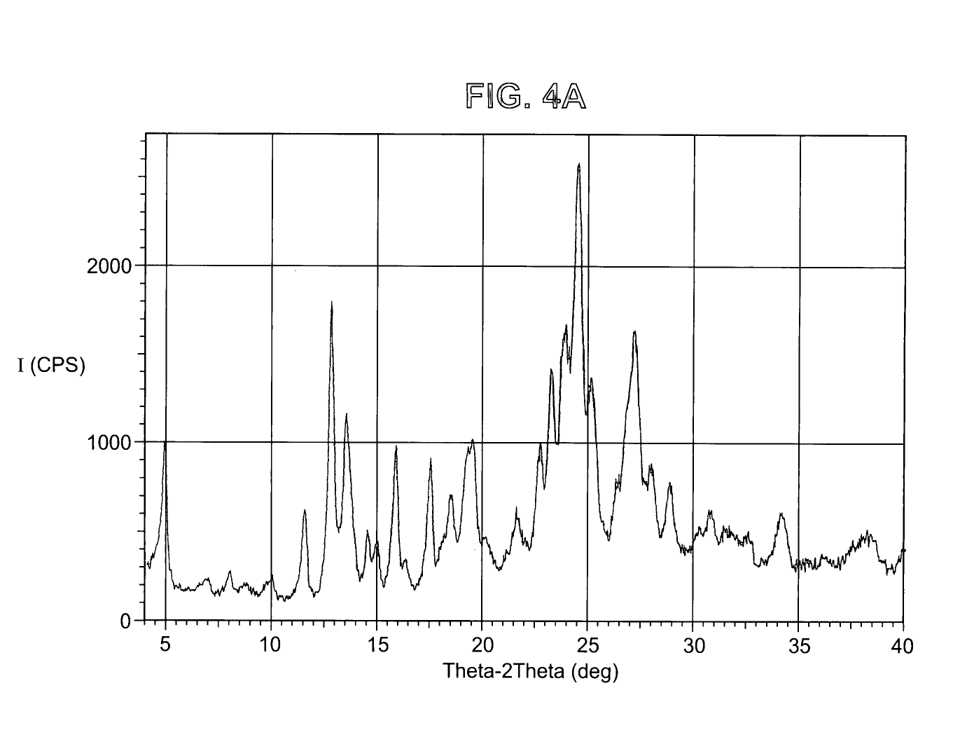

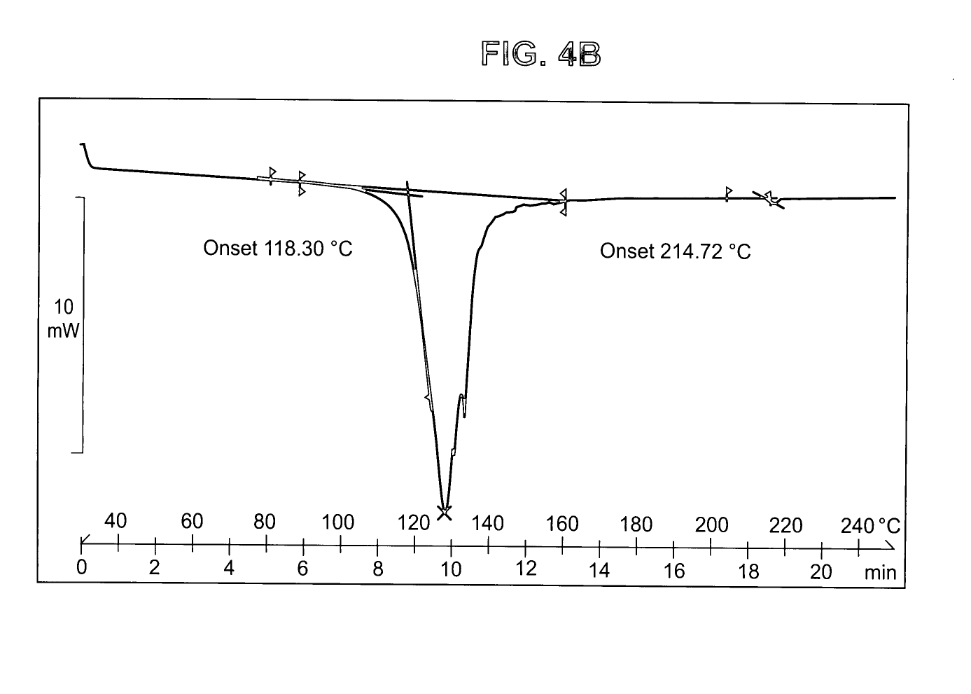

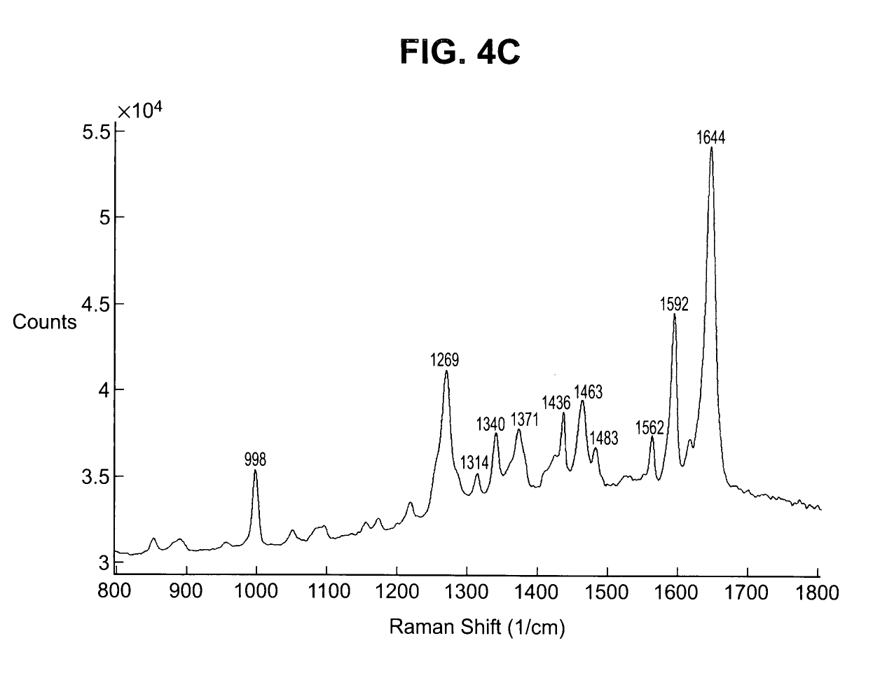

Form IV is characterized by an X-ray powder diffraction pattern with peaks at the following approximate diffraction angles (2θ): 4.85, 7.95, 9.85, 11.51, 12.80, 13.53, 14.56, 14.92, 15.80, 16.32, 17.43, 18.08, 18.44, 19.31, 20.08, 21.08, 21.61, 22.64, 23.24, 23.84, 24.48, 25.08, 26.24, 27.02, 27.92, 28.76, 30.12, 30.72, 31.40, 32.52, 34.07, 37.48, and 38.20. FIG. 4A provides an X-ray powder diffraction pattern for Form IV.

The DSC thermogram for Form IV, shown in FIG. 4B, indicates an onset of crystal melting endotherm at about 118° C., at a scan rate of 10° C./minute.

The Raman spectral diagram for Form IV, shown in FIG. 4C, includes Raman Shift peaks (cm⁻¹) at approximately 998, 1269, 1314, 1340, 1371, 1436, 1463, 1483, 1562, 1592, and 1644.

E. Polymorph Form V

Form V, an anhydrous form, can be prepared by slurrying Form IV solids in heavy mineral oil at 130° C., and then 180° C. for about 1.5 hours, followed by hexane wash and filtration. Form V has an aqueous solubility of about 8 μg/mL at pH 2 and about 0.2 μg/mL at pH 7.4. Form V is light-stable under ICH high intensity light conditions

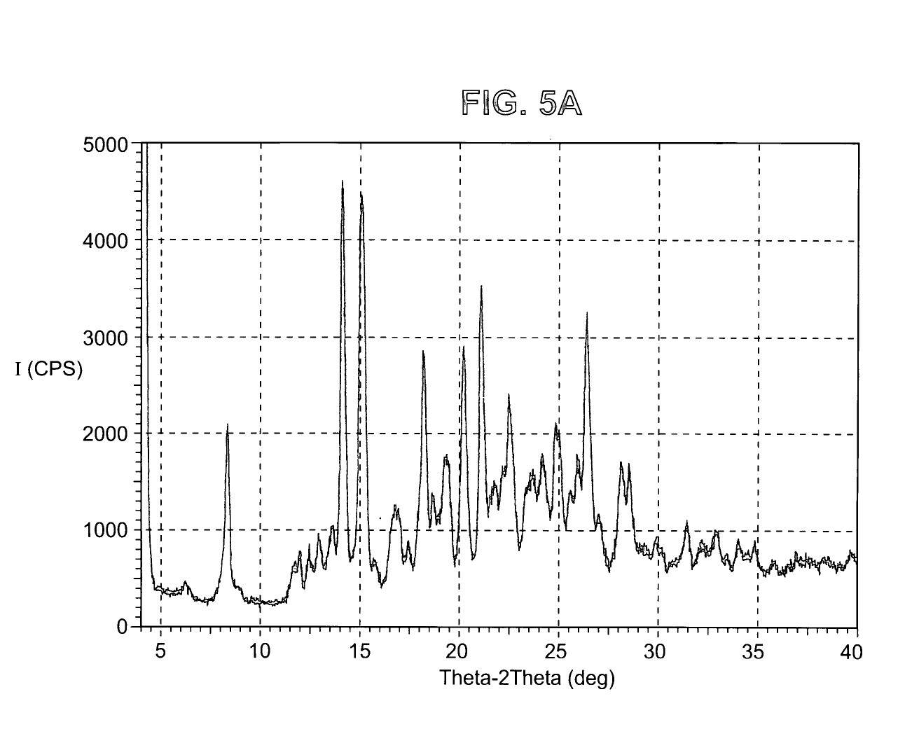

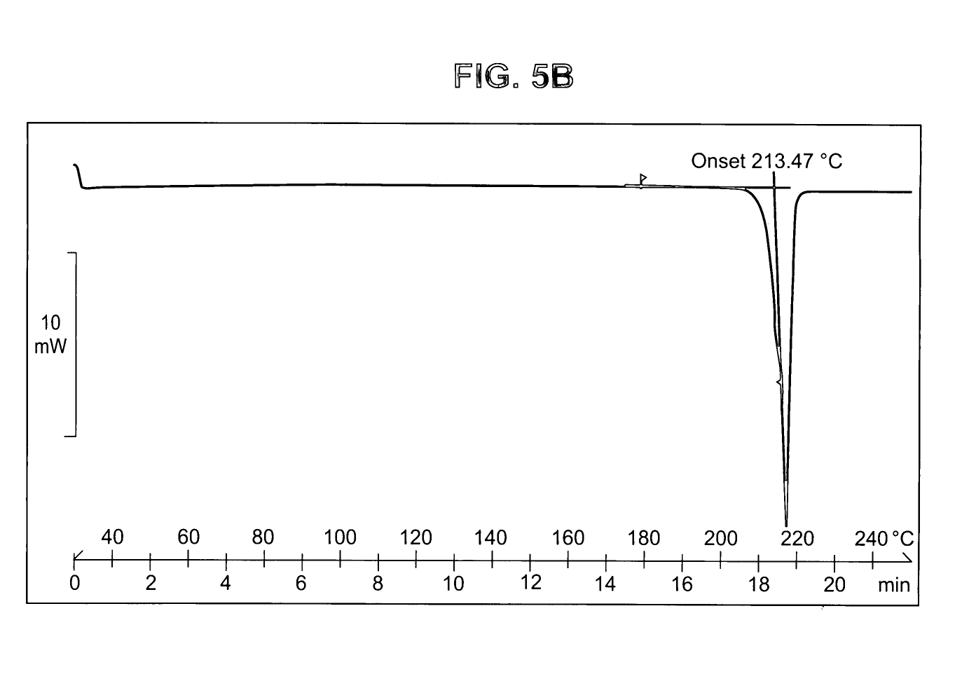

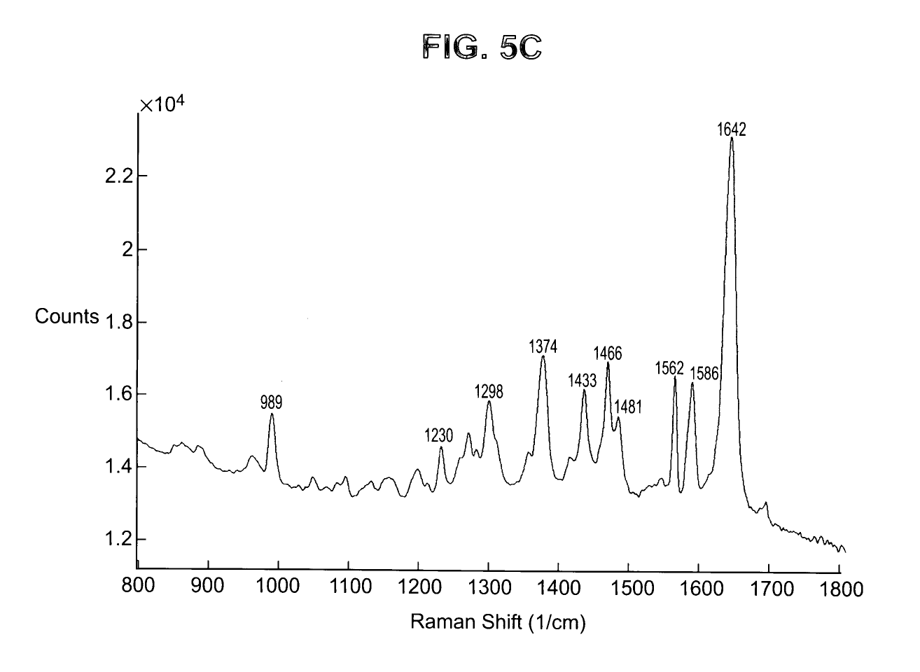

Form V is characterized by an X-ray powder diffraction pattern with peaks at the following approximate diffraction angles (2θ): 4.23, 8.38, 11.74, 12.00, 12.47, 12.95, 13.58, 14.17, 15.15, 16.76, 16.96, 17.44, 17.92, 18.28, 18.70, 19.37, 20.26, 21.16, 21.62, 21.84, 22.16, 22.54, 23.28, 23.64, 24.17, 24.84, 25.12, 25.58, 25.98, 26.48, 27.02, 28.16, 28.54, 29.14, 29.89, 31.40, 32.23, 32.66, and 39.68. FIG. 5A provides an X-ray powder diffraction pattern for Form V.

The DSC thermogram for Form V, shown in FIG. 5B, indicates an onset of crystal melting endotherm at about 210° C., at a scan rate of 1° C./minute.

The Raman spectral diagram for Form V, shown in FIG. 5C, includes Raman Shift peaks (cm⁻¹) at approximately 989, 1230, 1298, 1374, 1433, 1466, 1481, 1562, 1586, and 1642.

F. Polymorph Form Ia

Form Ia, which is a hydrate form, can be prepared by slurrying Form I in water at ambient temperature for seven days. Form Ia is light-stable under ICH high intensity light conditions.

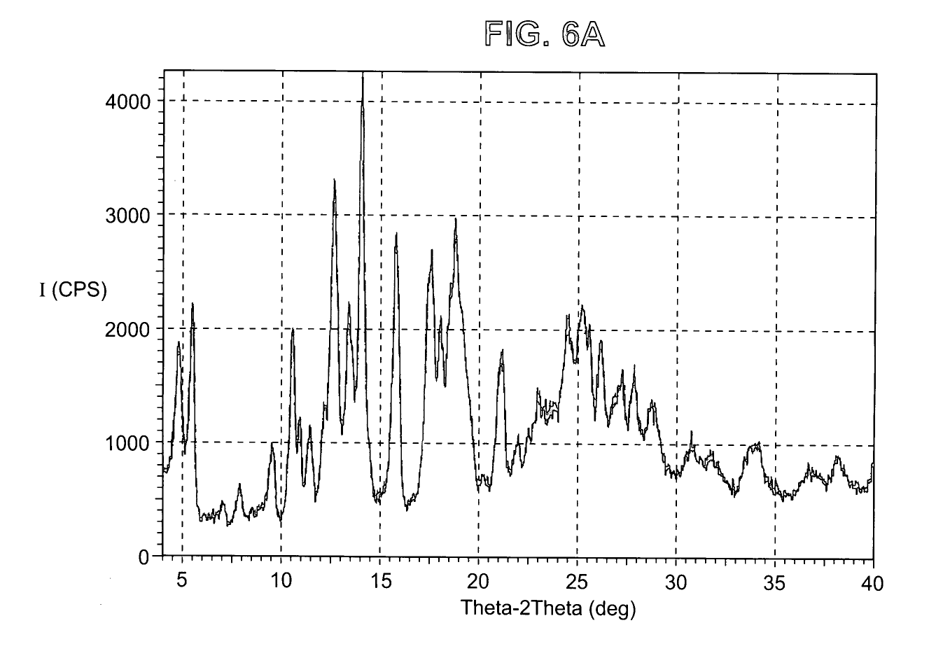

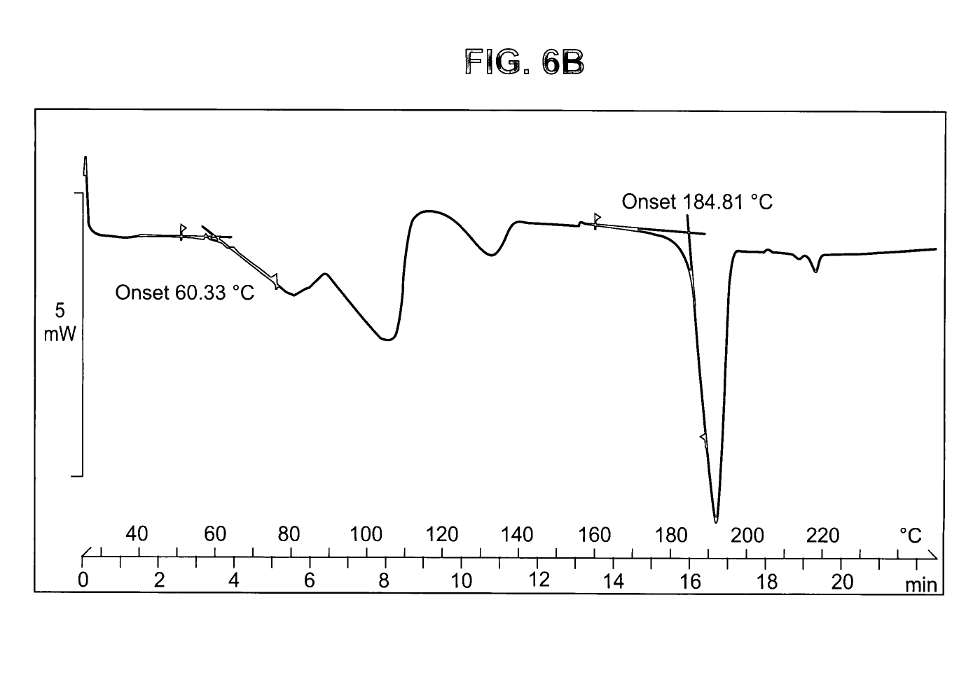

Form Ia is characterized by an X-ray powder diffraction pattern with peaks at the following approximate diffraction angles (2θ): 4.84, 5.49, 7.07, 7.90, 9.55, 10.60, 10.96, 11.48, 12.20, 12.72, 13.48, 14.10, 14.56, 15.78, 17.54, 18.08, 18.52, 18.88, 19.44, 21.11, 21.93, 22.48, 23.06, 23.72, 24.20, 24.48, 25.20, 25.56, 26.12, 26.72, 27.12, 27.78, 28.75, 30.36, 30.68, 31.20, 31.64, 32.04, 34.64, 34.97, 36.16, 36.60, 36.92, 37.24, 37.68, 38.12, 38.48, and 39.80. FIG. 6A provides an X-ray powder diffraction pattern for Form Ia.

The DSC thermogram for Form Ia, shown in FIG. 6B, indicates an onset of dehydration endotherm at about 60° C. and an onset of crystal melting endotherm at about 185° C., at a scan rate of 10° C./minute.

G. Polymorph Form Ib

Form Ib, which is a mono-hydrate, can be prepared by slurrying Form I in water at 90° C. for three days, or by crystallization from ethanol:water at greater than 65° C. Form Ib is physically and chemically stable for at least three months at 60° C. and 40° C./75% RH and is also light-stable under ICH high intensity light conditions.

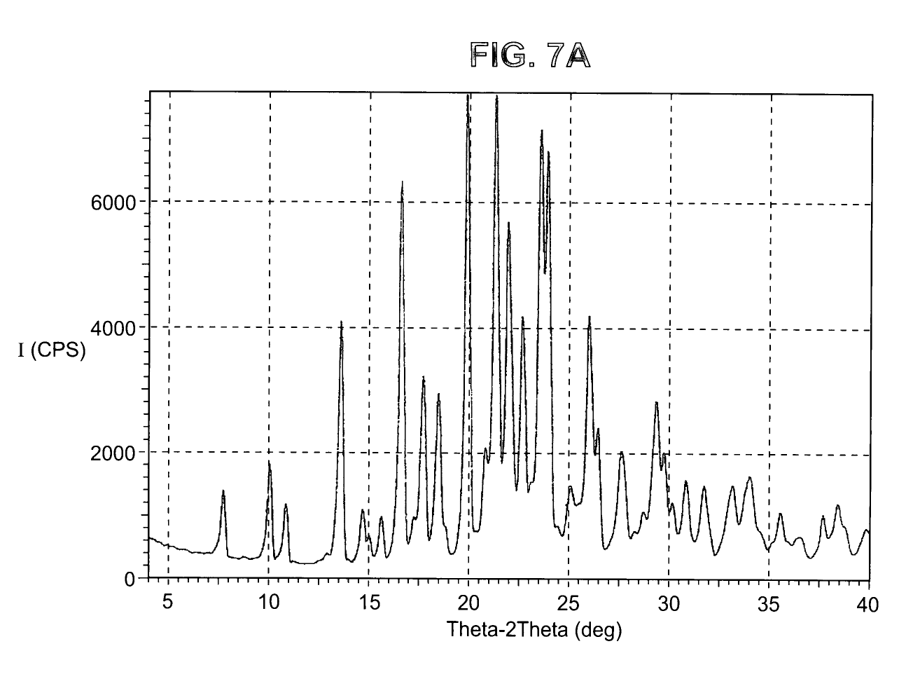

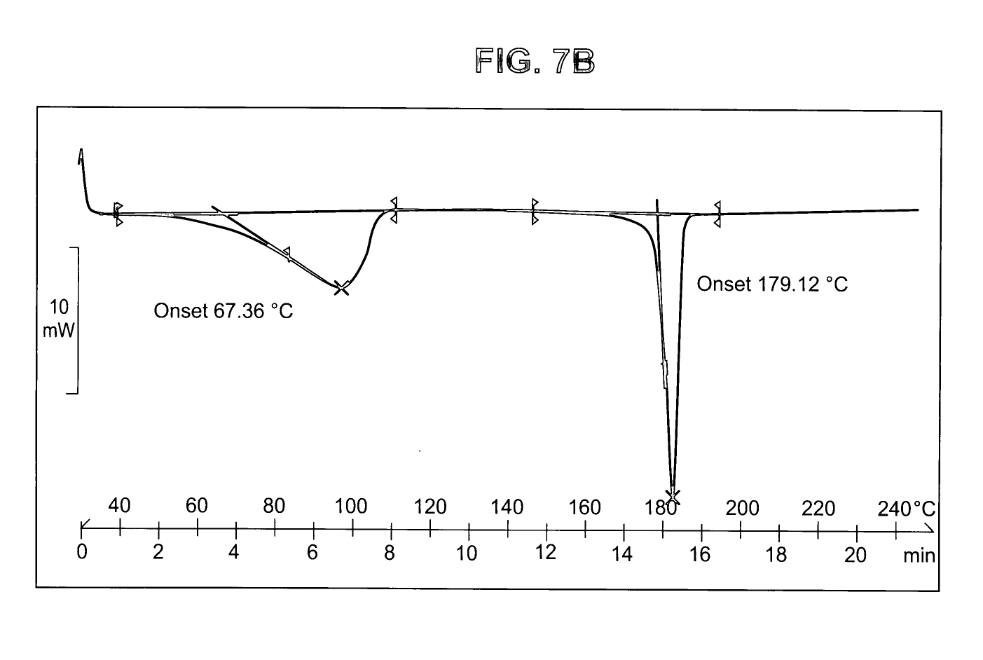

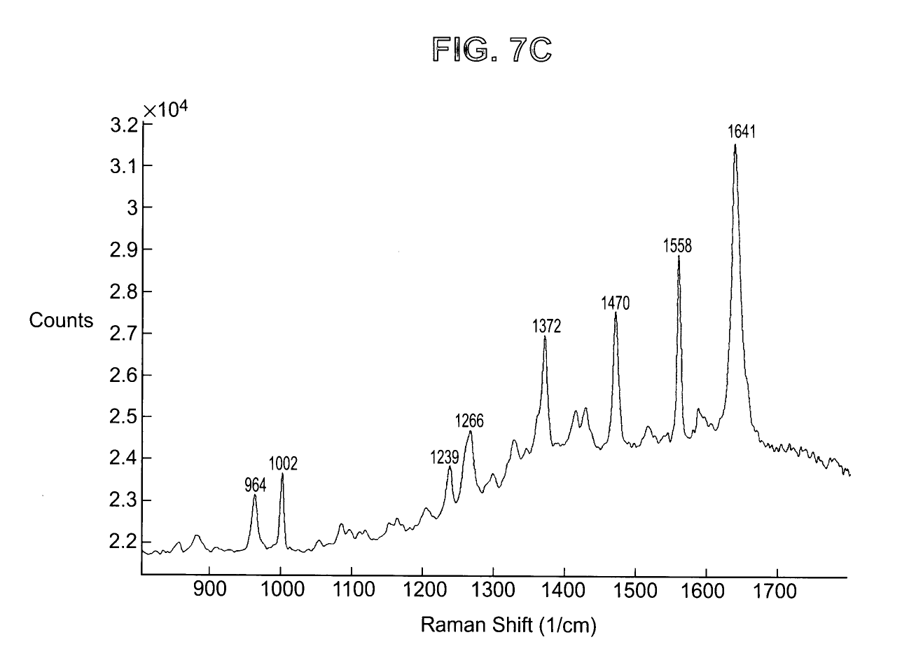

Form Ib is characterized by an X-ray powder diffraction pattern with peaks at the following approximate diffraction angles (2θ): 7.93, 10.23, 11.04, 13.12, 13.79, 14.88, 15.24, 15.81, 16.81, 17.40, 17.89, 18.64, 19.00, 20.11, 20.96, 21.53, 22.14, 22.87, 23.80, 24.16, 25.20, 26.20, 26.64, 27.76, 28.38, 28.84, 29.52, 29.92, 30.28, 30.92, 31.87, 32.80, 33.24, 34.07, 34.68, 35.74, 36.54, and 37.96. FIG. 7A provides an X-ray powder diffraction pattern for Form Ib.

The DSC thermogram for Form Ib, shown in FIG. 7B, indicates an onset of dehydration endotherm at about 67° C. and an onset of crystal melting endotherm at about 179° C., at a scan rate of 10° C./minute.

The Raman spectral diagram for Form Ib, shown in FIG. 7C, includes Raman Shift peaks (cm⁻¹) at approximately 964, 1002, 1239, 1266, 1372, 1470, 1558, and 1641.

H. Polymor Ph Form IIa

Form IIa, a mono-hydrate, can be prepared by slurrying Form II in water at ambient temperature for seven days. Form IIa is light-stable under ICH high intensity light conditions.

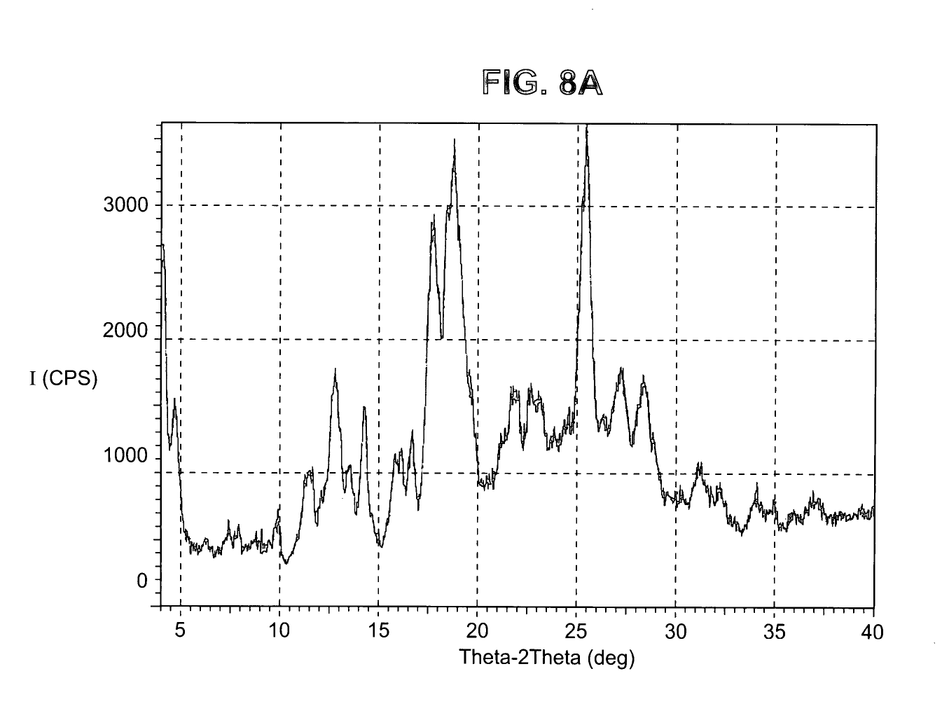

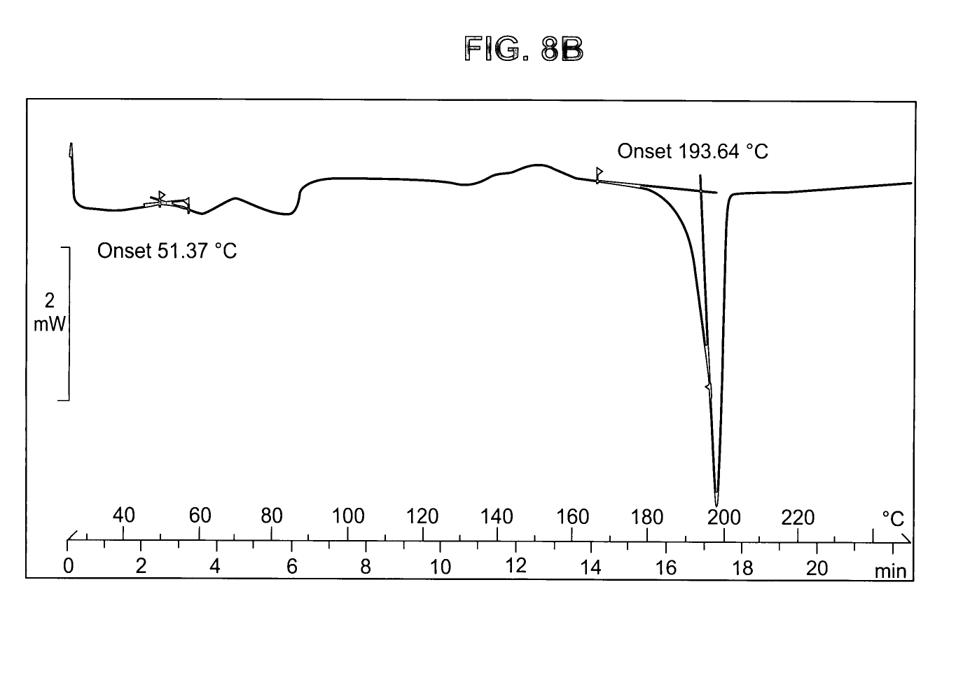

Form IIa is characterized by an X-ray powder diffraction pattern with peaks at the following approximate diffraction angles (2θ): 4.77, 7.64, 8.80, 9.82, 11.41, 12.75, 13.48, 14.23, 15.96, 16.64, 17.68, 18.76, 21.67, 22.85, 25.38, 27.16, 28.24, 30.12, 31.23, 32.16, 34.02, 34.80, 35.92, 36.92, 38.32, and 39.25. FIG. 8A provides an X-ray powder diffraction pattern for Form IIa.

The DSC thermogram for Form IIa, shown in FIG. 8B, indicates an onset of dehydration endotherm at about 51° C. and an onset of crystal melting endotherm at about 194° C., at a scan rate of 10° C./minute.

I. Polymorph Form IIb

Form IIb, a di-hydrate, can be prepared by slurrying Form II in water at 90° C. for three days and then ambient temperature for 17 days. Form IIb is light-stable under ICH high intensity light conditions.

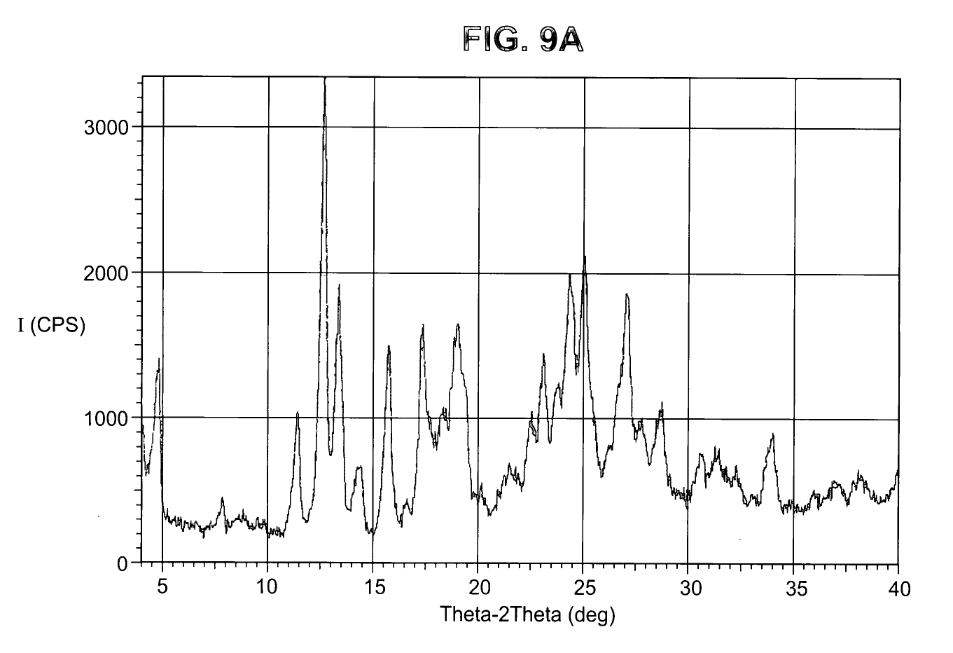

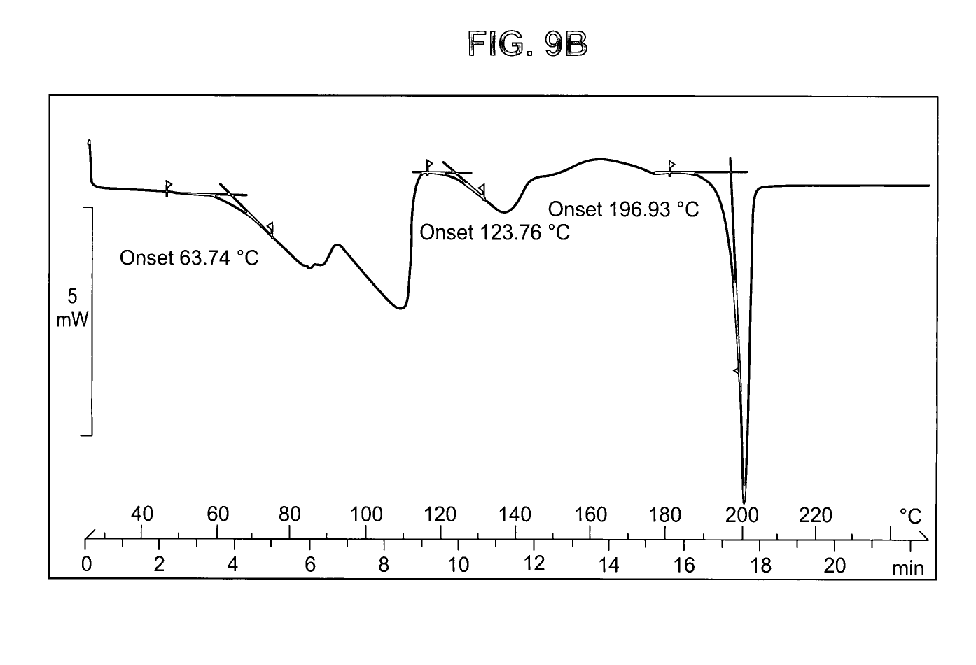

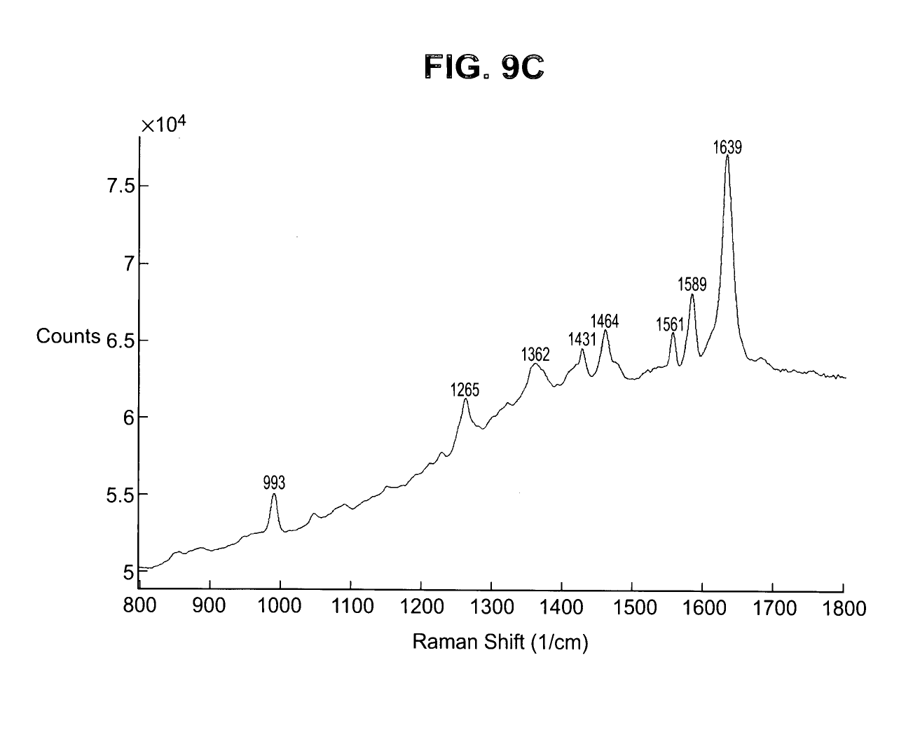

Form IIb is characterized by an X-ray powder diffraction pattern with peaks at the following approximate diffraction angles (2θ): 4.80, 7.86, 8.73, 11.44, 12.70, 13.41, 14.33, 15.71, 16.60, 17.43, 18.32, 19.03, 20.08, 21.56, 21.88, 22.56, 23.10, 23.76, 24.40, 25.04, 25.56, 26.20, 26.64, 27.02, 27.80, 28.64, 30.63, 31.36, 31.80, 32.28, 33.88, 35.95, 37.03, 37.80, 38.16, and 39.88. FIG. 9A provides an X-ray powder diffraction pattern for Form IIb.

The DSC thermogram for Form IIb, shown in FIG. 9B, indicates an onset of dehydration endotherm at about 64° C. and an onset of crystal melting endotherm at about 197° C., at a scan rate of 10° C./minute.

The Raman spectral diagram for Form IIb, shown in FIG. 9C, includes Raman Shift peaks (cm⁻¹) at approximately 993, 1265, 1362, 1431, 1464, 1561, 1589, and 1639.

J. Polymorph Form IIIa

Form IIIa, a di-hydrate, can be prepared by slurrying Form III in water at ambient temperature for seven days, or by placing Form III in 93% relative humidity at ambient temperature for ten days.

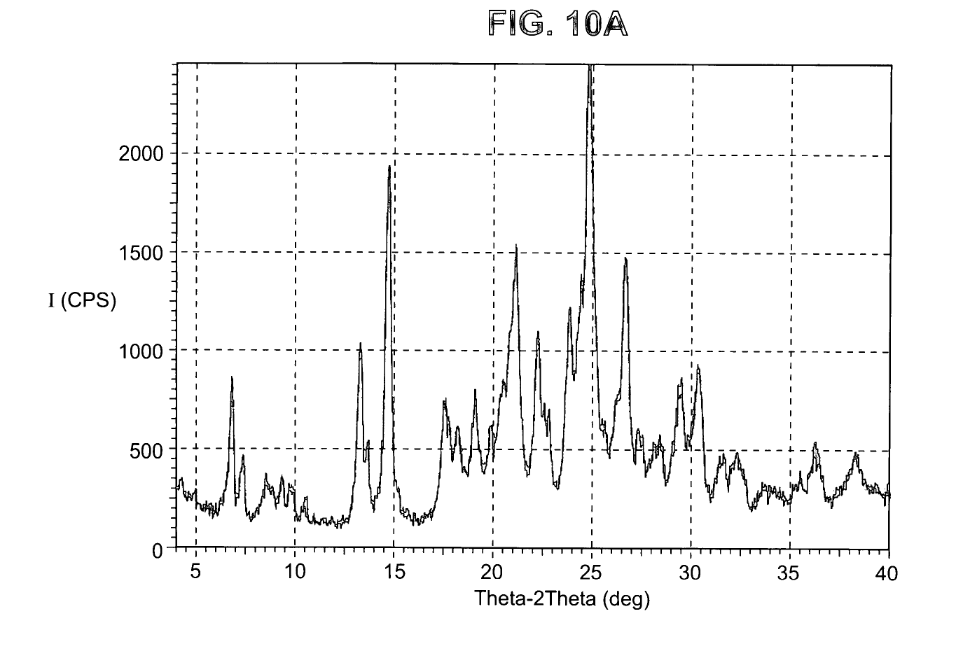

Form IIIa is characterized by an X-ray powder diffraction pattern with peaks at the following approximate diffraction angles (2θ): 6.81, 7.36, 8.71, 9.37, 9.80, 10.51, 13.31, 13.72, 14.72, 15.28, 17.60, 18.20, 19.09, 19.92, 20.48, 21.03, 22.27, 22.68, 23.84, 24.36, 24.86, 25.60, 26.16, 26.66, 27.33, 28.22, 29.41, 30.29, 31.48, 32.27, 33.60, 35.35, 36.22, and 38.21. FIG. 10A provides an X-ray powder diffraction pattern for Form IIa.

K. Polymorph Form IIIb

Form IIIb, an anhydrous form, can be prepared by drying Form IIIa at 50° C. under vacuum.

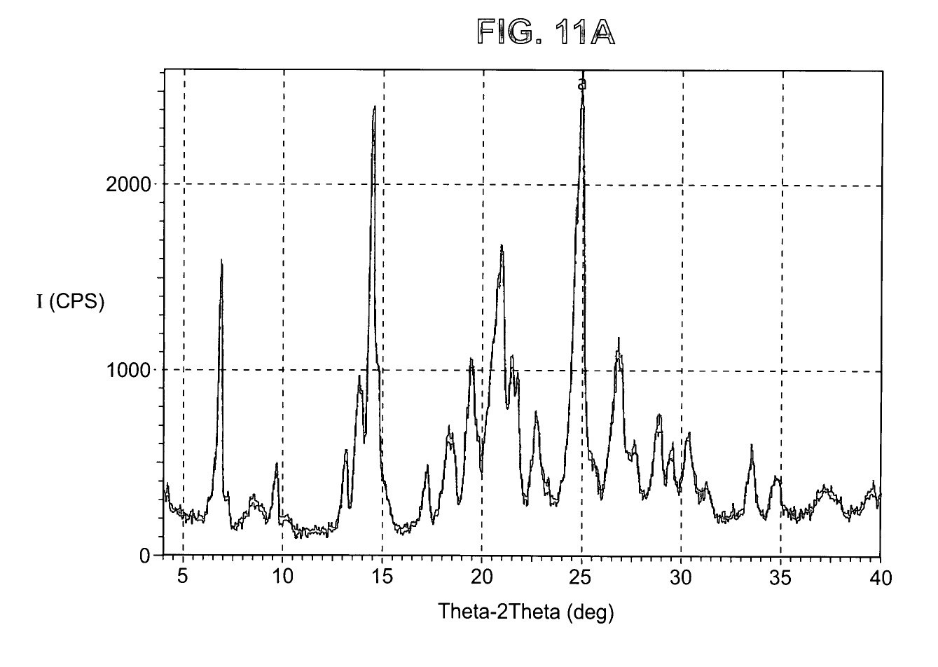

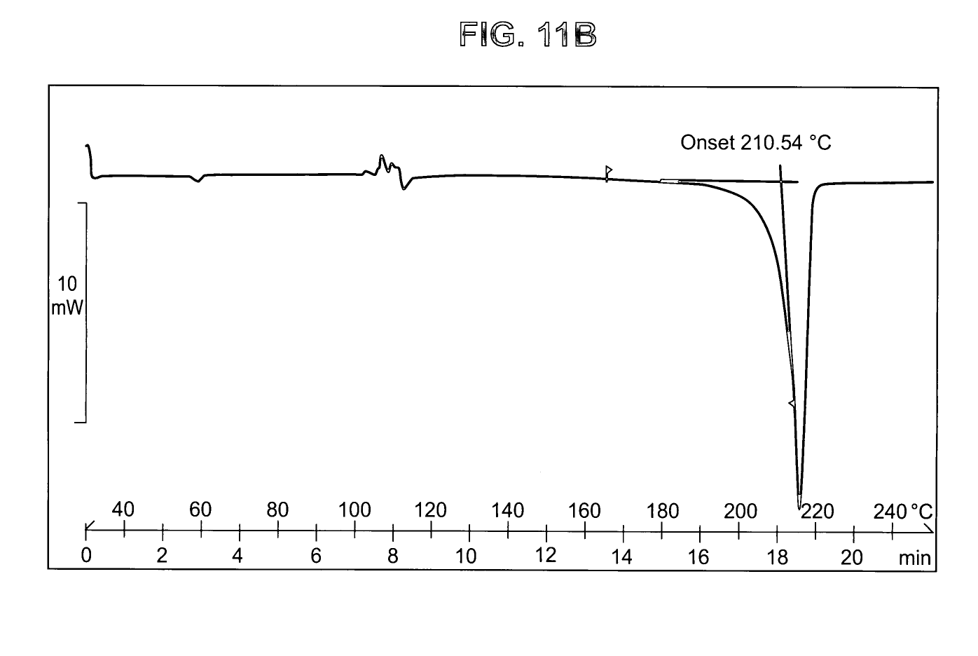

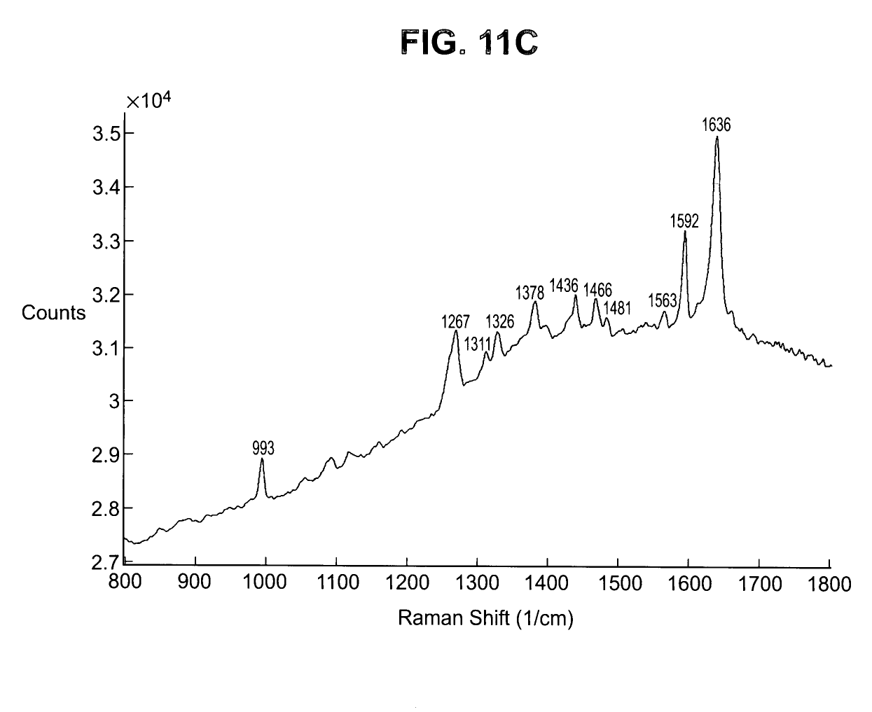

Form IIIb is characterized by an X-ray powder diffraction pattern with peaks at the following approximate diffraction angles (2θ): 6.28, 6.84, 7.36, 8.66, 9.66, 13.13, 13.80, 14.4718, 15.40, 17.21, 18.39, 19.46, 20.78, 21.56, 22.70, 24.81, 25.52, 26.79, 27.60, 28.80, 29.45, 30.32, 31.22, 33.47, 34.69, 37.16, 37.88, and 39.45. FIG. 11A provides an X-ray powder diffraction pattern for Form IIIb.

The DSC thermogram for Form IIIb, shown in FIG. 11B, indicates an onset of crystal melting endotherm at about 210° C., at a scan rate of 10° C./minute.

The Raman spectral diagram for Form IIIb, shown in FIG. 11C, includes Raman Shift peaks (cm⁻¹) at approximately 993, 1267, 1311, 1326, 1378, 1436, 1466, 1481, 1563, 1592, and 1636.

L. Polymorph Form IVa

Form IVa, a di-hydrate, can be prepared by slurrying Form IV in water for seven days. Form IVa is light-stable under ICH high intensity light conditions.

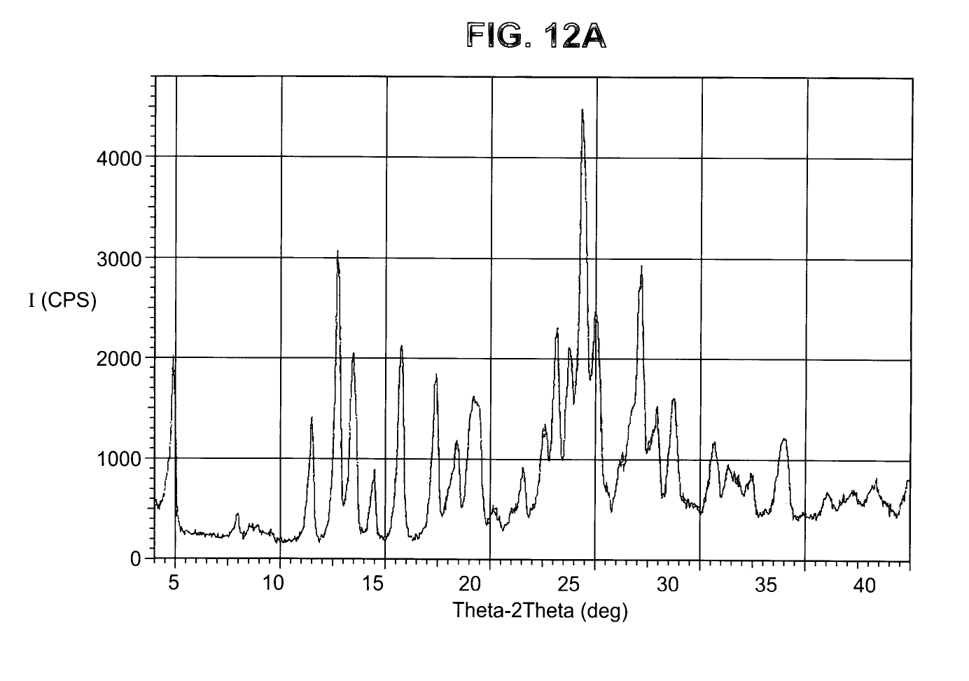

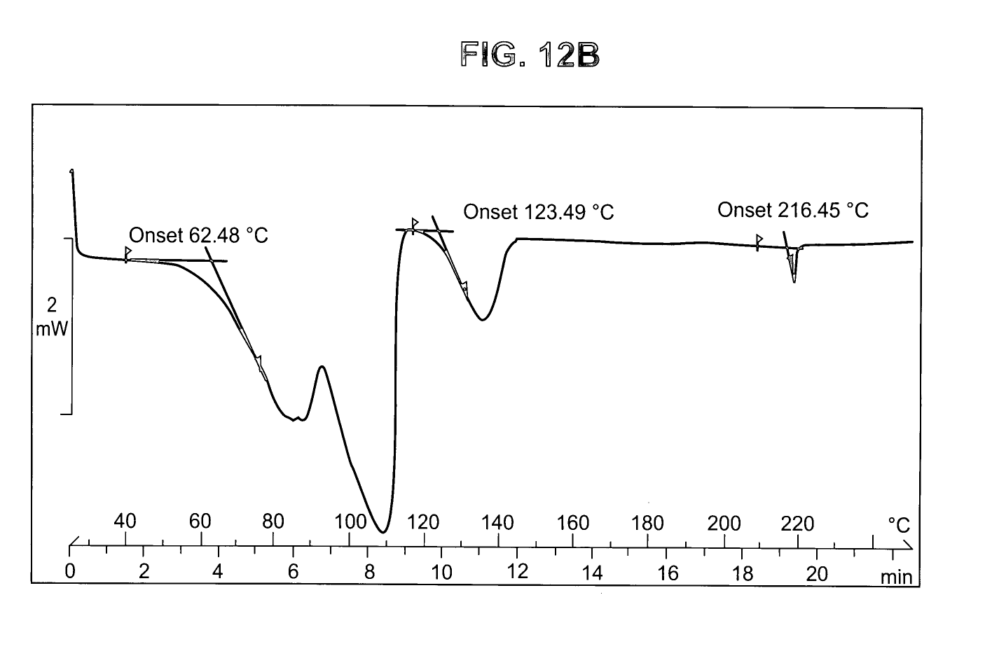

Form IVa is characterized by an X-ray powder diffraction pattern with peaks at the following approximate diffraction angles (2θ): 4.85, 7.95, 9.85, 11.51, 12.80, 13.53, 14.56, 14.92, 15.80, 16.32, 17.43, 18.08, 18.44, 19.31, 20.08, 21.08, 21.61, 22.64, 23.24, 23.84, 24.48, 25.08, 26.24, 27.02, 27.92, 28.76, 30.12, 30.72, 31.40, 32.52, 34.07, 37.48, and 38.20. FIG. 12A provides an X-ray powder diffraction pattern for Form IIb.

The DSC thermogram for Form IVa, shown in FIG. 12B, indicates an onset of dehydration endotherm at about 63° C. and an onset of crystal melting endotherm at about 123° C., at a scan rate of 10° C./minute.

M. Polymorph Form Va

Form Va, a di-hydrate form, can be prepared by slurrying Form V in water for seven days. Form Va is light-stable under ICH high intensity light conditions.

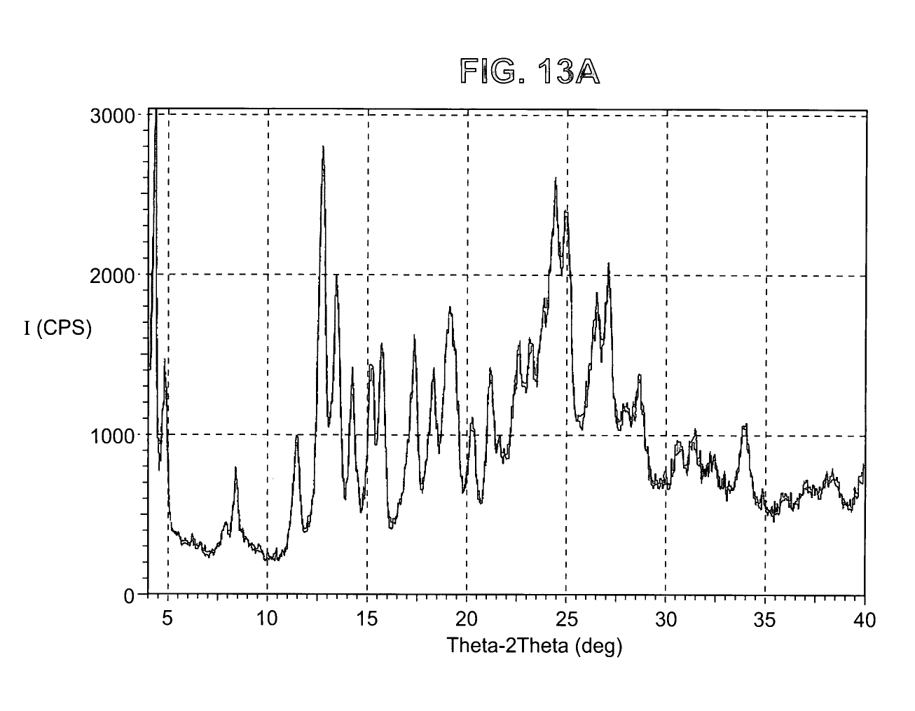

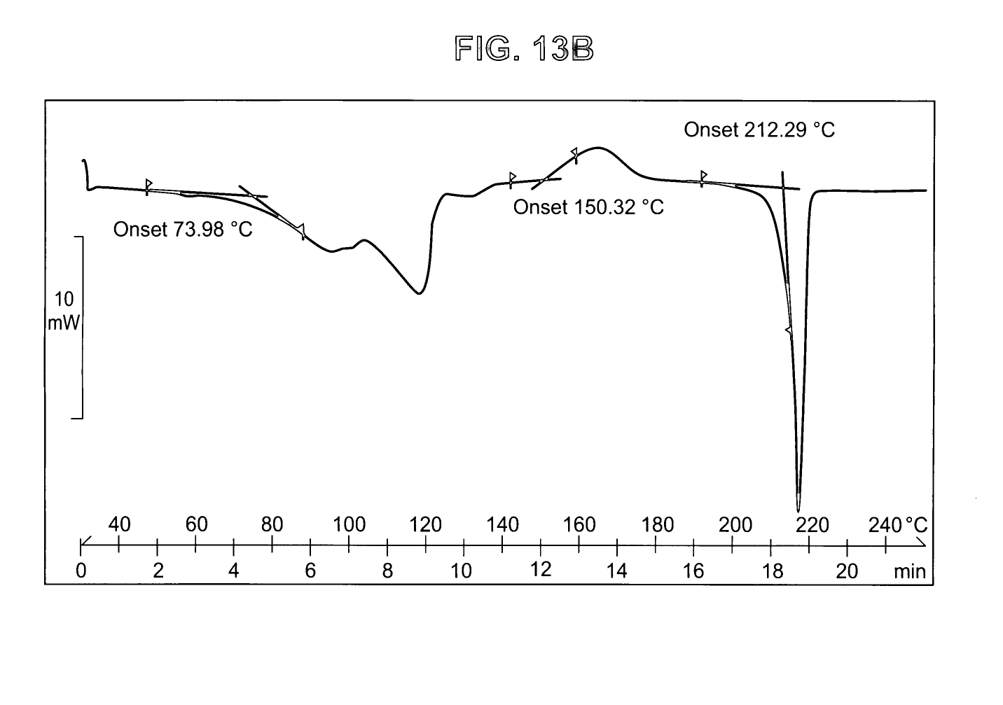

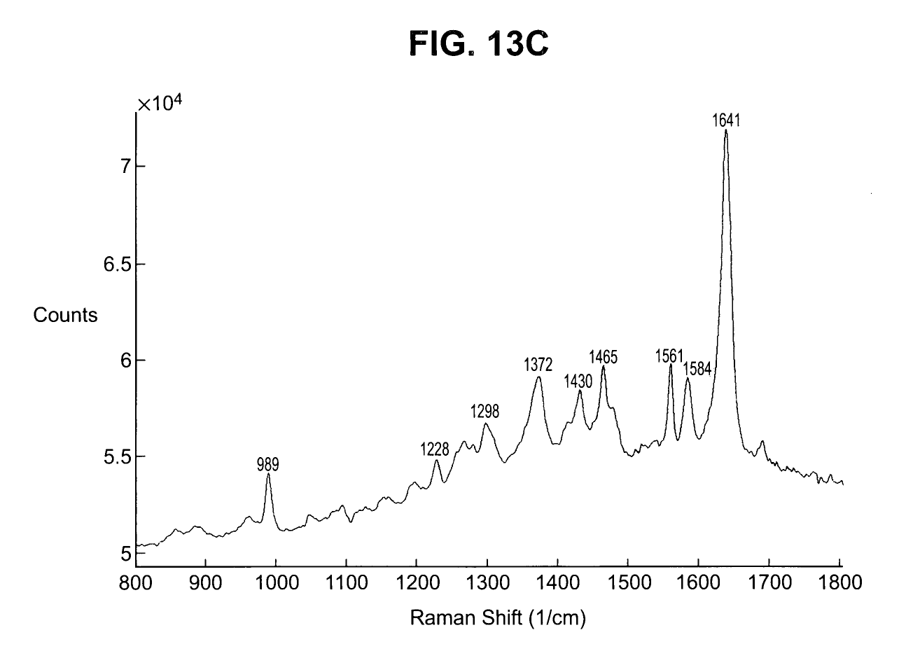

Form Va is characterized by an X-ray powder diffraction pattern with peaks at the following approximate diffraction angles (2θ): 4.26, 4.82, 7.92, 8.42, 8.96, 11.45, 12.70, 13.40, 14.21, 15.21, 15.70, 16.64, 16.96, 17.30, 18.28, 19.16, 20.24, 21.14, 21.60, 22.56, 23.20, 23.80, 24.44, 24.96, 26.60, 27.08, 27.96, 28.56, 29.04, 30.62, 31.34, 32.27, 32.84, 33.92, 34.83, 35.90, 36.99, and 37.44. FIG. 13A provides an X-ray powder diffraction pattern for Form Va.

The DSC thermogram for Form Va, shown in FIG. 13B, indicates an onset of dehydration endotherm at about 74° C. and an onset of crystal melting endotherm at about 211° C., at a scan rate of 10° C./minute.

The Raman spectral diagram for Form Va, shown in FIG. 13C, includes Raman Shift peaks (cm⁻¹) at approximately 989, 1228, 1298, 1372, 1430, 1465, 1561, 1584, and 1641.

N. Polymorph Form VI

Form VI, an anhydrous form, can be prepared by dehydration of Form Ib, such as by heating Form Ib at 140° C. for 10 minutes. Form VI is very hygroscopic and can be readily converted back to Form Ib under ambient humidity.

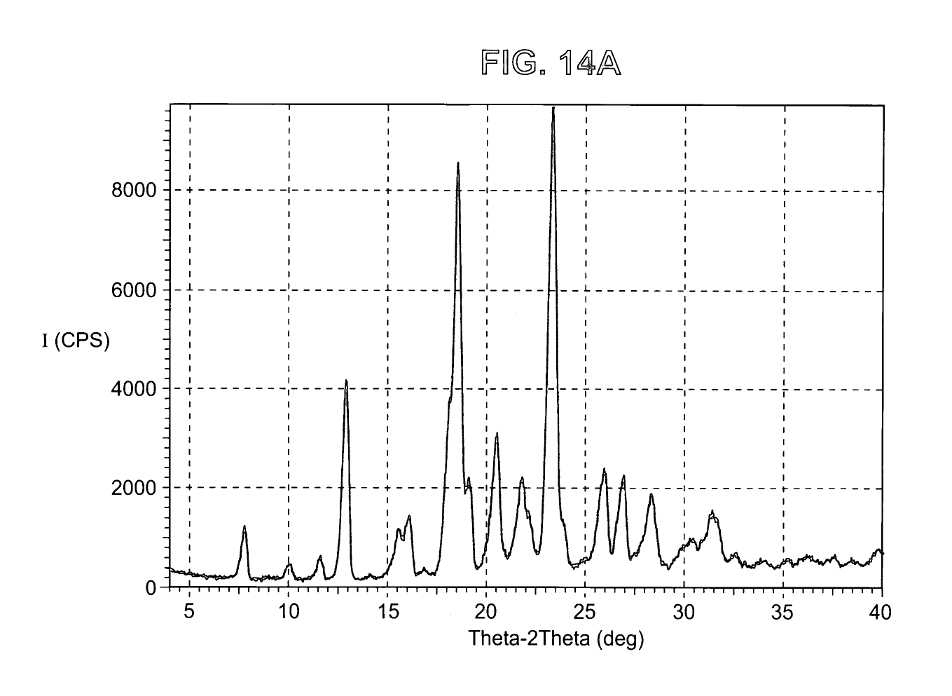

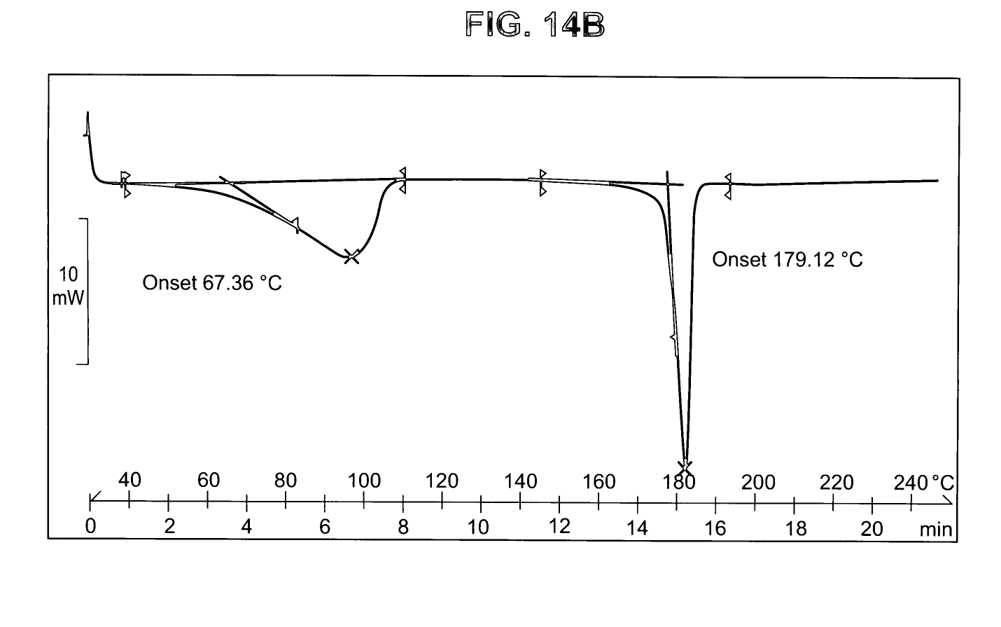

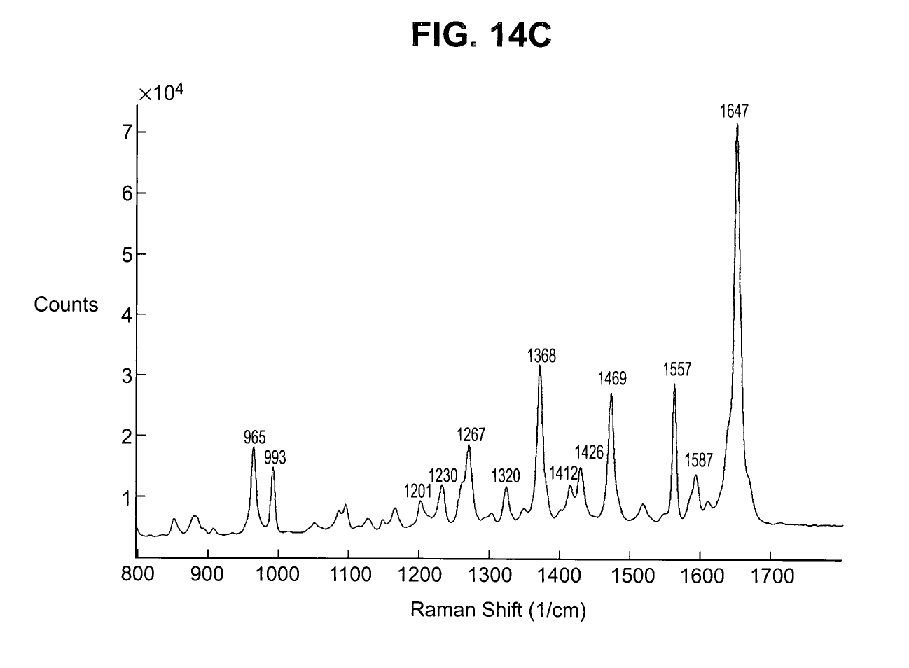

Form VI is characterized by an X-ray powder diffraction pattern with peaks at the following approximate diffraction angles (2θ): 7.74, 10.00, 11.56, 12.85, 15.56, 16.04, 17.80, 18.47, 19.20, 20.43, 21.72, 22.16, 23.28, 24.00, 25.83, 26.79, 28.23, 29.88, 30.36, 31.36, and 39.69. FIG. 14A provides an X-ray powder diffraction pattern for Form VI.

The DSC thermogram for Form VI, shown in FIG. 14B, indicates an onset of crystal melting endotherm at about 179° C., at a scan rate of 10° C./minute.

The Raman spectral diagram for Form VI, shown in FIG. 14C, includes Raman Shift peaks (cm⁻¹) at approximately: 965, 993, 1201, 1230, 1267, 1320, 1368, 1412, 1426, 1469, 1557, 1587, and 1647.

O. Amorphous Form

The amorphous form can be prepared by drop-wise dilution in water (approximately 1:10 ratio) of the compound of Formula I in polyethylene glycol 400 solution, or roto-vaporation of the compound of Formula I in methanol or THF solution, or lyophilization of the compound of Formula I in t-butanol solution.

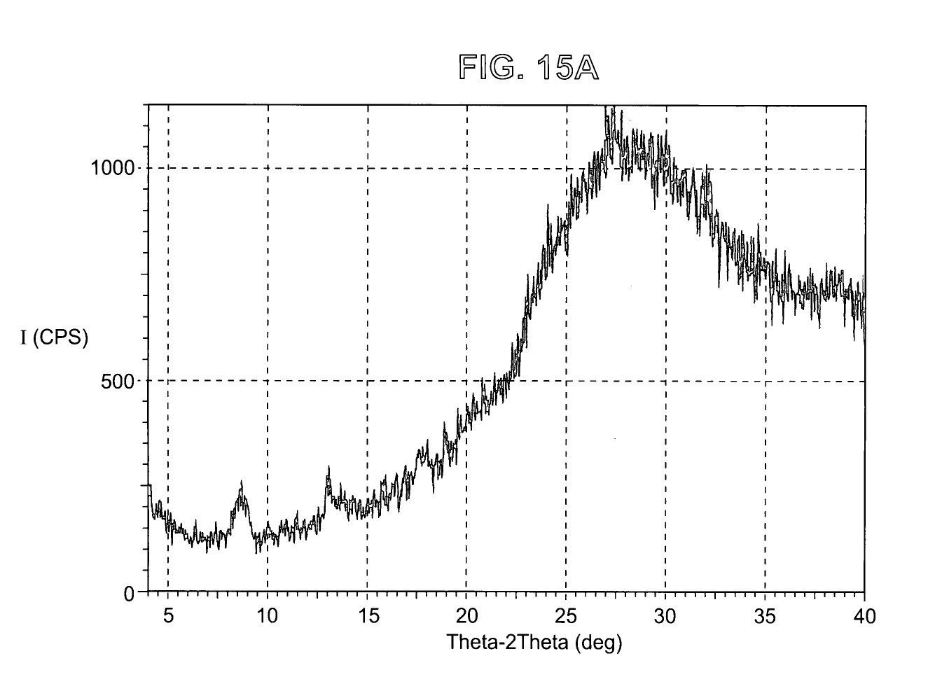

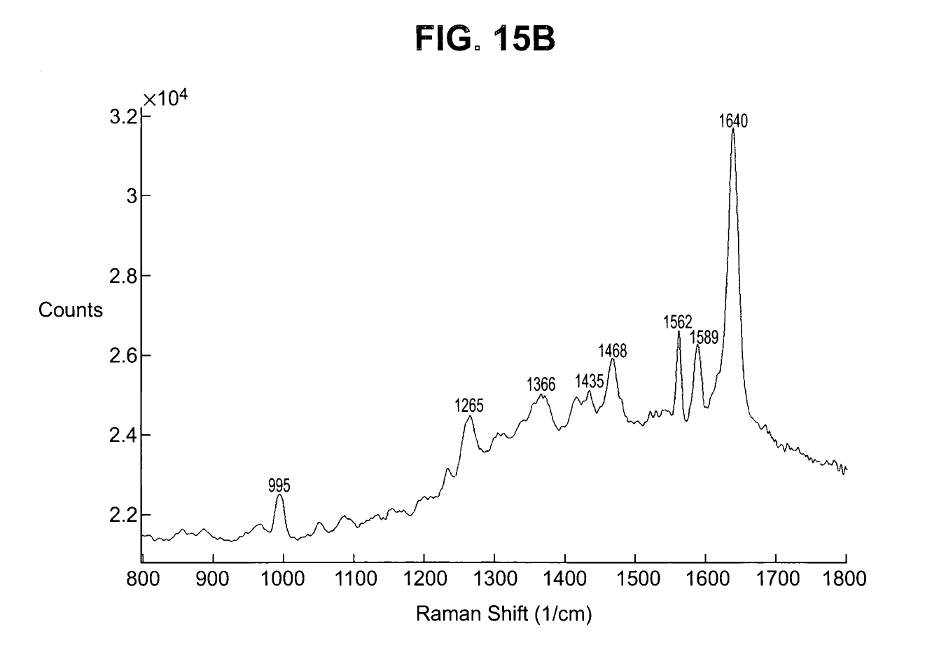

The X-ray powder diffraction pattern of the amorphous form is characterized by a typical amorphous broad hump-peak from 4 to 400, without any sharp peaks characteristic of crystalline forms. FIG. 15A provides an X-ray powder diffraction pattern for the amorphous form.

The Raman spectral diagram for the amorphous form, shown in FIG. 15B, includes Raman Shift peaks (cm⁻¹) at approximately 995, 1265, 1366, 1435, 1468, 1562, 1589, and 1640.

P. Mixtures

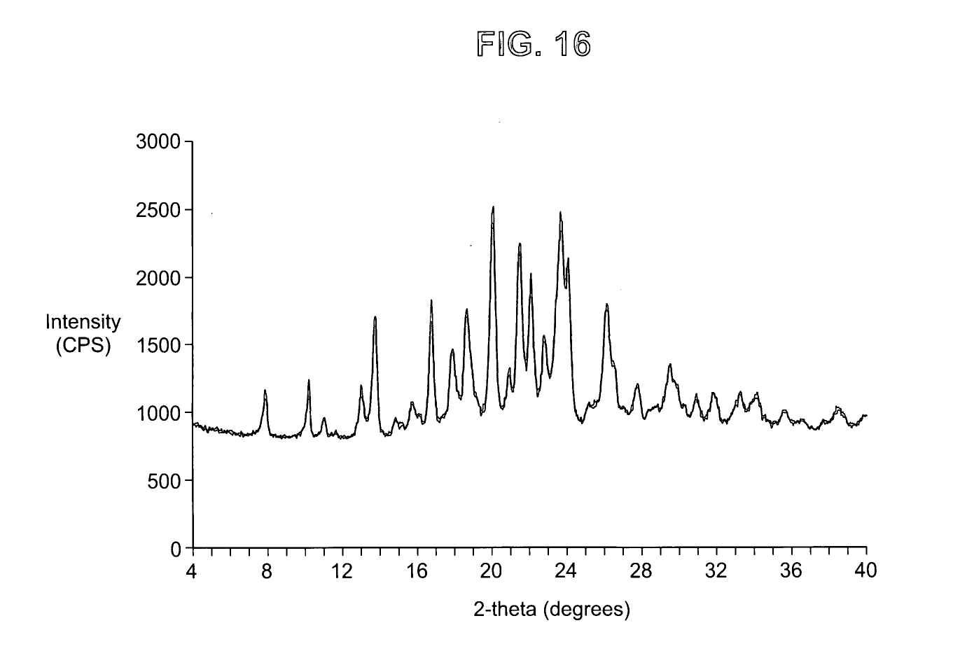

The crystalline and amorphous forms discussed above may also exist in mixtures, wherein the solid form exists as a mixture comprising at least two of the solid forms discussed above. For example, Form Ibm-2 is a meta-stable form that is a mixture of Forms Ib and VI. This meta-stable form can be prepared by dehydrating Form Ib under vacuum at temperatures of about 45° C. or greater. Partial hydration of Form VI will also result in the meta-stable Form Ibm-2. Form Ibm-2 will convert to Form Ib upon complete hydration under ambient humidity. Form Ibm-2 is characterized by an X-ray powder diffraction pattern with peaks as shown in FIG. 16. This diffraction pattern matches the pattern that results from addition of the diffraction patterns of Form Ib and Form VI. The DSC thermogram for Form lbm-2 indicates an onset of dehydration endotherm at about 73° C. and an onset of crystal melting endotherm at about 177° C., at a scan rate of 10° C./minute.

III. Pharmaceutical Compositions of the Invention

The active agents (i.e., the polymorph or amorphous forms, or mixtures thereof, of the ompound of Formula I described herein) of the invention may be formulated into pharmaceutical compositions suitable for both veterinary and human medical use. Pharmaceutical compositions of the invention comprise a therapeutically effective amount of the active agent and one or more inert, pharmaceutically acceptable carriers, and optionally any other therapeutic ingredients, stabilizers, or the like. The carrier(s) must be pharmaceutically acceptable in the sense of being compatible with the other ingredients of the formulation and not unduly deleterious to the recipient thereof. The compositions may further include diluents, buffers, binders, disintegrants, thickeners, lubricants, preservatives (including antioxidants), flavoring agents, taste-masking agents, inorganic salts (e.g., sodium chloride), antimicrobial agents (e.g., benzalkonium chloride), sweeteners, antistatic agents, surfactants (e.g., polysorbates such as “TWEEN 20” and “TWEEN 80”, and pluronics such as F68 and F88, available from BASF), sorbitan esters, lipids (e.g., phospholipids such as lecithin and other phosphatidylcholines, phosphatidylethanolamines, fatty acids and fatty esters, steroids (e.g., cholesterol)), and chelating agents (e.g., EDTA, zinc and other such suitable cations). Other pharmaceutical excipients and/or additives suitable for use in the compositions according to the invention are listed in “Remington: The Science & Practice of Pharmacy” 19^{th} ed., Williams & Williams, (1995), and in the “Physician's Desk Reference”, 52^{nd} ed., Medical Economics, Montvale, N.J. (1998), and in “Handbook of Pharmaceutical Excipients”, Third Ed., Ed. A. H. Kibbe, Pharmaceutical Press, 2000. The active agents of the invention may be formulated in compositions including those suitable for oral, rectal, topical, nasal, ophthalmic, or parenteral (including intraperitoneal, intravenous, subcutaneous, or intramuscular injection) administration.

The amount of the active agent in the formulation will vary depending upon a variety of factors, including dosage form, the condition to be treated, target patient population, and other considerations, and will generally be readily determined by one skilled in the art. A therapeutically effective amount will be an amount necessary to modulate, regulate, or inhibit a protein kinase. In practice, this will vary widely depending upon the particular active agent, the severity of the condition to be treated, the patient population, the stability of the formulation, and the like. Compositions will generally contain anywhere from about 0.001% by weight to about 99% by weight active agent, preferably from about 0.01% to about 5% by weight active agent, and more preferably from about 0.01% to 2% by weight active agent, and will also depend upon the relative amounts of excipients/additives contained in the composition.

A pharmaceutical composition of the invention is administered in conventional dosage form prepared by combining a therapeutically effective amount of an active agent as an active ingredient with one or more appropriate pharmaceutical carriers according to conventional procedures. These procedures may involve mixing, granulating and compressing or dissolving the ingredients as appropriate to the desired preparation.

The pharmaceutical carrier employed may be either a solid or liquid. Exemplary solid carriers include lactose, sucrose, talc, gelatin, agar, pectin, acacia, magnesium stearate, stearic acid and the like. Exemplary liquid carriers include syrup, peanut oil, olive oil, water and the like. Similarly, the carrier may include time-delay or time-release materials known in the art, such as glyceryl monostearate or glyceryl distearate alone or with a wax, ethylcellulose, hydroxypropylmethylcellulose, methylmethacrylate and the like.

A variety of pharmaceutical forms can be employed. Thus, if a solid carrier is used, the preparation can be tableted, placed in a hard gelatin capsule in powder or pellet form or in the form of a troche or lozenge. The amount of solid carrier may vary, but generally will be from about 25 mg to about 1 g. If a liquid carrier is used, the preparation can be in the form of syrup, emulsion, soft gelatin capsule, sterile injectable solution or suspension in an ampoule or vial or non-aqueous liquid suspension.

To obtain a stable water-soluble dose form, a pharmaceutically acceptable salt of an active agent is dissolved in an aqueous solution of an organic or inorganic acid, such as 0.3M solution of succinic acid or citric acid. If a soluble salt form is not available, the active agent may be dissolved in a suitable cosolvent or combinations of cosolvents. Examples of suitable cosolvents include, but are not limited to, alcohol, propylene glycol, polyethylene glycol 300, polysorbate 80, gylcerin and the like in concentrations ranging from 0-60% of the total volume. The composition may also be in the form of a solution of a salt form of the active agent in an appropriate aqueous vehicle such as water or isotonic saline or dextrose solution.

It will be appreciated that the actual dosages of the active agents used in the compositions of this invention will vary according to the particular complex being used, the particular composition formulated, the mode of administration and the particular site, host and disease being treated. Those skilled in the art using conventional dosage-determination tests in view of the experimental data for an agent can ascertain optimal dosages for a given set of conditions. For oral administration, an exemplary daily dose generally employed is from about 0.001 to about 1000 mg/kg of body weight, more preferably from about 0.001 to about 50 mg/kg body weight, with courses of treatment repeated at appropriate intervals. Administration of prodrugs is typically dosed at weight levels that are chemically equivalent to the weight levels of the fully active form.

The compositions of the invention may be manufactured in manners generally known for preparing pharmaceutical compositions, e.g., using conventional techniques such as mixing, dissolving, granulating, dragee-making, levigating, emulsifying, encapsulating, entrapping or lyophilizing. Pharmaceutical compositions may be formulated in a conventional manner using one or more physiologically acceptable carriers, which may be selected from excipients and auxiliaries that facilitate processing of the active compounds into preparations that can be used pharmaceutically.

Proper formulation is dependent upon the route of administration chosen. For injection, the agents of the invention may be formulated into aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration intranasally or by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contains suitable stabilizers or agents that increase the solubility of the compounds to allow for the preparation of highly concentrated solutions.

For administration to the eye, the active agent is delivered in a pharmaceutically acceptable ophthalmic vehicle such that the compound is maintained in contact with the ocular surface for a sufficient time period to allow the compound to penetrate the corneal and internal regions of the eye, including, for example, the anterior chamber, posterior chamber, vitreous body, aqueous humor, vitreous humor, cornea, iris/cilary, lens, choroid/retina and selera. The pharmaceutically acceptable ophthalmic vehicle may be, for example, an ointment, vegetable oil, or an encapsulating material. A compound of the invention may also be injected directly into the vitreous and aqueous humor or subtenon.

Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides.

In addition to the formulations described above, the compounds may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt.

A pharmaceutical carrier for hydrophobic compounds is a cosolvent system comprising benzyl alcohol, a nonpolar surfactant, a water-miscible organic polymer, and an aqueous phase. The cosolvent system may be a VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) contains VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

IV. Method of Using the Compounds of the Invention

The inventive compounds are useful for mediating the activity of protein kinases. More particularly, the compounds are useful as anti-angiogenesis agents and as agents for modulating and/or inhibiting the activity of protein kinases, such as the activity associated with VEGF, FGF, CDK complexes, TEK, CHK1, LCK, FAK, and phosphorylase kinase among others, thus providing treatments for cancer or other diseases associated with cellular proliferation mediated by protein kinases in mammals, including humans.

Therapeutically effective amounts of the agents of the invention may be administered, typically in the form of a pharmaceutical composition, to treat diseases mediated by modulation or regulation of protein kinases. An “effective amount” is intended to mean that amount of an agent that, when administered to a mammal in need of such treatment, is sufficient to effect treatment for a disease mediated by the activity of one or more protein kinases, such as tyrosine kinases. Thus, a therapeutically effective amount of a compound of the invention is a quantity sufficient to modulate, regulate, or inhibit the activity of one or more protein kinases such that a disease condition that is mediated by that activity is reduced or alleviated. The effective amount of a given compound will vary depending upon factors such as the disease condition and its severity and the identity and condition (e.g., weight) of the mammal in need of treatment, but can nevertheless be routinely determined by one skilled in the art. “Treating” is intended to mean at least the mitigation of a disease condition in a mammal, such as a human, that is affected, at least in part, by the activity of one or more protein kinases, such as tyrosine kinases, and includes: preventing the disease condition from occurring in a mammal, particularly when the mammal is found to be predisposed to having the disease condition but has not yet been diagnosed as having it; modulating and/or inhibiting the disease condition; and/or alleviating the disease condition. Exemplary disease conditions include diabetic retinopathy, neovascular glaucoma, rheumatoid arthritis, psoriasis, age-related macular degeneration (AMD), and cancer (solid tumors).

The activity of the inventive compounds as modulators of protein kinase activity, such as the activity of kinases, may be measured by any of the methods available to those skilled in the art, including in vivo and/or in vitro assays. Examples of suitable assays for activity measurements include those described in Parast C. et al., BioChemistry, 37, 16788-16801 (1998); Jeffrey et al., Nature, 376, 313-320 (1995); WIPO International Publication No. WO 97/34876; and WIPO International Publication No. WO 96/14843.

### V. EXAMPLES

The following examples are given to illustrate the invention, but should not be considered as limitations of the invention. Unless otherwise indicated, all temperatures are set forth in degrees Celsius and all parts and percentages are by weight. HPLC data was obtained using a Hewlett Packard HP-1100 HPLC.

### Example 1

Polymorph Form I

The compound of Formula I crude material from synthesis (155 mg) was slurried in 5 mL ethanol and then heated to reflux for 30 min. The sample was allowed to slowly cool down to 23° C. The solids were collected by filtration and dried at 85° C. under high vacuum. Form I was confirmed by X-ray diffraction and HPLC purity was >98%.

### Example 2

Polymorph Form II

Form I from Example 1 was dissolved in tetrahydrofuran at 60° C. and then re-crystallized by gradual addition of hexanes to obtain Form II. Form II was confirmed by X-ray diffraction (HPLC purity >98%).

### Example 3

Polymorph Form III

Form I from Example 1 was slurried in light mineral oil at 192° C. for 1 hour and then cooled down to room temperature. The solids were collected by filtration, washed with hexanes, and then dried at 50° C. under vacuum. Form III was confirmed by X-ray diffraction (HPLC purity >97%).

### Example 4

Polymorph Form IV

The compound of Formula I crude material from synthesis was§dissolved in ethyl acetate and ethanol. Re-crystallization was done by addition of 1:1 NaHCO₃:Water. Form IV was confirmed by X-ray diffraction (HPLC purity>99%).

### Example 5

Polymorph Form V

Form IV solids from Example 4 were suspended in heavy mineral oil at 130° C. and then slurried at 180° C. for one and a half hours. The solids were collected by filtration, washed with hexanes, and then dried under vacuum. Form V was confirmed by X-ray diffraction (HPLC purity >99%).

### Example 6

Polymorph Form Ia

Form I from Example 1 was slurried in water (approximately 20-40 mg/mL) at ambient temperature for seven days to obtain Form Ia. Form Ia was confirmed by X-ray diffraction (HPLC purity >99%).

### Example 7

Polymorph Form Ib

Form I from Example 1 was slurried in water (approximately 20-40 mg/mL) at 90° C. for three days to obtain Form Ib. Alternatively, Form Ib was obtained by crystallization from ethanol:water at 65° C. Form Ib was confirmed by X-ray diffraction (HPLC purity >99%).

### Example 8

Polymorph Form Ha

Form II from Example 2 was slurried in water (approximately 20-40 mg/mL) at ambient temperature for seven days to obtain Form IIa. Form IIa was confirmed by X-ray diffraction (HPLC purity >99%).

### Example 9

Polymorph Form IIb

Form II from Example 2 was slurried in water (approximately 20-40 mg/mL) at 90° C. for three days and then ambient temperature for 17 days to obtain Form IIb. Form IIb was confirmed by X-ray diffraction.

### Example 10

Polymornh Form IIIa

Form III from Example 3 was placed in 93% relative humidity at ambient temperature for ten days or slurried in water (approximately 20-40 mg/mL) at ambient temperature for seven days to obtain Form IIIa. Form IIIa was confirmed by X-ray diffraction.

### Example 11

Polymorph Form IIIb

Form IIIa from Example 10 was dried at 50° C. under vacuum to obtain Form IIIb. Form IIIa was confirmed by X-ray diffraction.

### Example 12

Polymorph Form IVa

Form IV from Example 4 was slurried in water (approximately 20-40 mg/mL) at ambient temperature for seven days to obtain Form IVa. Form IVa was confirmed by X-ray diffraction and DSC.

### Example 13

Polymorph Form Va

Form V from Example 5 was slurried in water (approximately 20-40 mg/mL) at ambient temperature for seven days to obtain Form Va. Form Va was confirmed by X-ray diffraction.

### Example 14

Polymorph Form VI

Form Ib from Example 7 was heated at 140° C. for 10 minutes to obtain Form VI.

Form VI was confirmed by X-ray diffraction.

### Example 15

Amorphous Form

Amorphous form was prepared by drop-wise dilution in water (approximately 1:10 ratio) of the compound of Formula I in polyethylene glycol 400 solution, or prepared by rotary evaporation of the compound of Formula I in methanol or THF solution, or prepared by lyophilization of the compound of Formula I in t-butanol solution.

### Example 16

Polymorph Form Ibm-2

Form Ib from Example 7 was heated at 50° C. under vacuum to obtain Form Ibm-2, which was confirmed by X-ray diffraction to be a mixture of polymorph Form Ib and Form VI.

### Example 17

Use of Polymorph Form Ib in Hyaluronate Suspension Formulation

Sodium hyaluronate (1% w/w) is dissolved in pH 7.4 isotonic sodium phosphate buffer solution containing 0.85% sodium chloride, 0.022% dibasic sodium phosphate, 0.004% mono-basic sodium phosphate, and 97.15% water to form a viscous solution. The solution is then sterilized by filtration. Micronized and sterilized Form Ib from Example 7 (0.1-1.0% w/w) is then added to form a homogeneous suspension by gentle impeller mixing.

### Example 18

Use of Polymor Ph Form Ib in CMC Suspension Formulation

Sodium carboxymethylcellulose (CMC) (0.5% w/w) is dissolved in water and then sterilized by filtration. An appropriate amount of Form Ib from Example 7 (0.1-1.0% w/w) is then added to form a homogenous suspension by vortexing and sonicating up to 10 minutes.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. A crystalline form of 2,5-dimethyl-2H-pyrazole-3-carboxylic acid {2-fluoro-5-[3-((E)-2-pyridin-2-yl-vinyl)-1H-indazol-6-ylamino]-phenyl}-amide, or a pharmaceutically acceptable salt thereof.

2. The crystalline form of claim 1, wherein the crystalline form is a substantially pure polymorph of any of Forms I, II, III, IV, V, Ia, Ib, IIa, IIb, IIIa, IIIb, IVa, Va or VI.

3. The crystalline form of claim 1, wherein the crystalline form is a substantially pure polymorph of Form Ib.

4. The crystalline form of claim 3, wherein the crystalline form has an X-ray powder diffraction pattern comprising peaks at diffraction angles (2θ) of 10.2 and 13.8.

5. The crystalline form of claim 3, wherein the crystalline form has an X-ray powder diffraction pattern comprising peaks at diffraction angles (2θ) of 10.2, 13.8, 20.1, and 26.2.

6. The crystalline form of claim 3, wherein the crystalline form has an X-ray powder diffraction pattern comprising peaks at diffraction angles (2θ) essentially the same as shown in FIG. 7A.

7. The crystalline form of claim 3, wherein the crystalline form has a Raman spectra essentially the same as shown in FIG. 7C.

8. The crystalline form of claim 1, wherein the crystalline form is substantially a pure polymorph of Form I, wherein the crystalline form has an X-ray powder diffraction pattern comprising peaks at diffraction angles (2θ) essentially the same as shown in FIG. 1A, and a Raman spectra essentially the same as shown in FIG. 1C.

9. The crystalline form of claim 1, wherein the crystalline form is substantially a pure polymorph of Form II, wherein the crystalline form has an X-ray powder diffraction pattern comprising peaks at diffraction angles (2θ) essentially the same as shown in FIG. 2A, and a Raman spectra essentially the same as shown in FIG. 2C.

10. The crystalline form of claim 1, wherein the crystalline form is substantially a pure polymorph of Form III, wherein the crystalline form has an X-ray powder diffraction pattern comprising peaks at diffraction angles (2θ) essentially the same as shown in FIG. 3A, and a Raman spectra essentially the same as shown in FIG. 3C.

11. The crystalline form of claim 1, wherein the crystalline form is substantially a pure polymorph of Form IV, wherein the crystalline form has an X-ray powder diffraction pattern comprising peaks at diffraction angles (2θ) essentially the same as shown in FIG. 4A, and a Raman spectra essentially the same as shown in FIG. 4C.

12. The crystalline form of claim 1, wherein the crystalline form is substantially a pure polymorph of Form V, wherein the crystalline form has an X-ray powder diffraction pattern comprising peaks at diffraction angles (2θ) essentially the same as shown in FIG. 5A, and a Raman spectra essentially the same as shown in FIG. 5C.

13. The crystalline form of claim 1, wherein the crystalline form is substantially a pure polymorph of Form Ia, wherein the crystalline form has an X-ray powder diffraction pattern comprising peaks at diffraction angles (2θ) essentially the same as shown in FIG. 6A.

14. The crystalline form of claim 1, wherein the crystalline form is substantially a pure polymorph of Form IIa, wherein the crystalline form has an X-ray powder diffraction pattern comprising peaks at diffraction angles (2θ) essentially the same as shown in FIG. 8A.

15. The crystalline form of claim 1, wherein the crystalline form is substantially a pure polymorph of Form IIb, wherein the crystalline form has an X-ray powder diffraction pattern comprising peaks at diffraction angles (2θ) essentially the same as shown in FIG. 9A, and a Raman spectra essentially the same as shown in FIG. 9C.

16. The crystalline form of claim 1, wherein the crystalline form is substantially a pure polymorph of Form IIIa, wherein the crystalline form has an X-ray powder diffraction pattern comprising peaks at diffraction angles (2θ) essentially the same as shown in FIG. 10A.

17. The crystalline form of claim 1, wherein the crystalline form is substantially a pure polymorph of Form IIIb, wherein the crystalline form has an X-ray powder diffraction

18. pattern comprising peaks at diffraction angles (2θ) essentially the same as shown in FIG. 11A, and a Raman spectra essentially the same as shown in FIG. 11C.

19. The crystalline form of claim 1, wherein the crystalline form is substantially a pure polymorph of Form IVa, wherein the crystalline form has an X-ray powder diffraction pattern comprising peaks at diffraction angles (2θ) essentially the same as shown in FIG. 12A.

20. The crystalline form of claim 1, wherein the crystalline form is substantially a pure polymorph of Form Va, wherein the crystalline form has an X-ray powder diffraction pattern comprising peaks at diffraction angles (2θ) essentially the same as shown in FIG. 13A, and a Raman spectra essentially the same as shown in FIG. 13C.

21. The crystalline form of claim 1, wherein the crystalline form is substantially a pure polymorph of Form VI, wherein the crystalline form has an X-ray powder diffraction pattern comprising peaks at diffraction angles (2θ) essentially the same as shown in FIG. 14A, and a Raman spectra essentially the same as shown in FIG. 14C.

22. An amorphous form of 2,5-dimethyl-2H-pyrazole-3-carboxylic acid-{2-fluoro-5-[3-((E)-2-pyridin-2-yl-vinyl)-1H-indazol-6-ylamino]-phenyl}-amide, or a pharmaceutically acceptable salt thereof, wherein the amorphous form has an X-ray powder diffraction pattern comprising peaks at diffraction angles (2θ) essentially the same as shown in FIG. 15A, and a Raman spectra essentially the same as shown in FIG. 15B.

23. A solid form of 2,5-dimethyl-2H-pyrazole-3-carboxylic acid (2-fluoro-5-[3-((E)-2-pyridin-2-yl-vinyl)-1H-indazol-6-ylamino]-phenyl)-amide, wherein the solid form is a mixture comprising at least two of the following solid forms: polymorph Forms I, II, III, IV, V, Ia, Ib, IIa, IIb, IIIa, IIIb, IVa, Va, VI, and an amorphous form.

24. The crystalline form of claim 1, wherein the crystalline form is a substantially pure polymorph of Form Ibm-2, wherein the crystalline form has an X-ray powder diffraction pattern comprising peaks at diffraction angles (2θ) essentially the same as shown in FIG. 16.

25. A pharmaceutical composition comprising any of the crystalline, solid or amorphous forms of claims 1 to 23.

26. A method of treating a mammalian disease condition mediated by protein kinase activity, comprising administering to a mammal in need thereof a therapeutically effective amount of the pharmaceutical composition of claim 24.

27. A method according to claim 25, wherein the mammalian disease condition is associated with tumor growth, cell proliferation, or angiogenesis.

28. A method of modulating the activity of a protein kinase receptor, comprising contacting the kinase receptor with an effective amount of any of the crystalline, solid or amorphous form of claims 1 to 23.

29. The method according to claim 27, wherein the protein kinase receptor is a VEGF receptor.
